(19) United States
(12) Patent Application Publication
(10) Pub. No.: US 2025/0049889 A1
(43) Pub. Date: Feb. 13, 2025

(54) NEUROPROTECTIVE COMPOSITIONS AND USE THEREOF

(71) Applicant: NanoNeuron Therapeutics, Inc., Natick, MA (US)

(72) Inventors: Qiang Feng, Natick, MA (US); Gina Elsen, Natick, MA (US); Po-Hsuen Chen, Natick, MA (US); Shi-Jiang Lu, Natick, MA (US)

(21) Appl. No.: 18/930,790

(22) Filed: Oct. 29, 2024

Related U.S. Application Data
(63) Continuation of application No. 17/265,158, filed on Feb. 1, 2021.
(60) Provisional application No. 62/713,055, filed on Aug. 1, 2018.

(51) Int. Cl. A61K 38/18; A61K 9/51; A61K 38/20; A61K 38/21; A61K 38/22; A61P 25/28

(52) U.S. Cl. CPC A61K 38/185; A61K 9/5161; A61K 38/204; A61K 38/212; A61K 38/215; A61K 38/217; A61K 38/2257; A61P 25/28

(57) ABSTRACT

The present disclosure relates generally to biocompatible nanoparticles, and in particular, neuroprotective nanoparticles comprising ciliary neurotrophic factor and/or oncostatin M. Methods for making and using the same are also provided.

### CROSS-REFERENCE TO RELATED APPLICATIONS

This application claims priority to and the benefit of U.S. Provisional Application No. 62/713,055 filed Aug. 1, 2018, the entire disclosure of which is incorporated herein by reference.

### FIELD

The present disclosure relates generally to biocompatible nanoparticles, in particular, neuroprotective nanoparticles comprising neurotrophic factors such as ciliary neurotrophic factor and/or oncostatin M. Methods for making and using the same are also provided.

### BACKGROUND

The retina is the inner sheath of the eye; it is a complex system that transforms a light influence into neural impulsation and transfers the latter to the brain. The structure of the retina and main types of its cells are very similar in all vertebrates. The degeneration and death of retinal ganglion cells (RGC) and optic nerve fibers, which project to the retina from cerebral visual centers, and photoreceptors that are neurosensory light-sensitive cells generating electrical signals in response to their illumination, are the leading causes of blindness in human.

Optic neuropathy is a generic term for optic nerve diseases, which lead to irreversible vision loss due to optic nerve damage and death of RGCs. Glaucoma is the most common form of optic neuropathy and a leading cause of blindness affecting 70 million people worldwide. Currently, no treatment is available to reverse glaucomatous RGC damage or vision loss. In clinic, glaucoma is diagnosed by optic disc pallor, central depression, excavation and an increased cup-to-disc ratio. Many patients with glaucoma display an elevated intraocular pressure (IOP), but they do not normally experience blurry vision and visual field loss until a late stage of the disease. Treatment for glaucoma is solely directed at lowering the IOP through pharmacological, surgical or laser-based approaches. Although these treatments slow down the disease progression, they do not repair the damaged optic nerve or reverse vision loss. Therefore, new treatment strategies for glaucoma are at demand and neuroprotection focused on improving neuron survival and preventing progressive RGC damage is critical.

Rod and cone photoreceptors, which comprise the retinal outer nuclear layer, are the light sensing cells of the eye. They convert light signals into electrical impulses, initiating the visual transduction cascade which sends visual information to be processed in the brain. Mammalian photoreceptors do not have the capacity to regenerate, and when lost due to injury or disease, light is no longer perceived. Macular degeneration including retinitis pigmentosa, Stargardt disease and age-related macular degeneration are characterized by progressive loss of photoreceptors, and is the leading cause of vision loss—more than cataracts and glaucoma combined. At present, macular degeneration is considered an incurable and irreversible eye disease that ultimately leads to untreatable blindness, no effective treatment to restore visual function or halt disease progression.

Thus, a need exists for effective treatment for optic as well as other neuronal degeneration.

### SUMMARY

The present disclosure provides, inter alia, compositions and methods for use in the treatment of a disease or condition.

One aspect relates to a composition for use in the treatment of a disease or condition in a subject in need thereof, comprising: a plurality of nanoparticles comprising dextran sulfate and chitosan; and an effective amount of a neurotrophic factor associated with the nanoparticles, wherein preferably the neurotrophic factor is selected from the group consisting of Brain-derived neurotrophic factor (BDNF), Nerve growth factor (NGF), Neurotrophin-3 (NT-3), Neurotrophin-4 (NT-4), Ciliary neurotrophic factor (CNTF), leukemia inhibitory factor (LIF), interleukin-6 (IL-6), prolactin, growth hormone, leptin, interferons (i.e., interferon-α, -β, and -γ), oncostatin M (OSM), Glial cell line-derived neurotrophic factor (GDNF), Artemin, Neurturin, Persephin, and Ephrins.

In another aspect, a method for treating a disease or condition is provided, comprising administering to a subject in need thereof a pharmaceutical composition, wherein said pharmaceutical composition comprises:
a plurality of nanoparticles comprising dextran sulfate and chitosan;
an effective amount of a neurotrophic factor, preferably selected from the group consisting of Brain-derived neurotrophic factor (BDNF), Nerve growth factor (NGF), Neurotrophin-3 (NT-3), Neurotrophin-4 (NT-4), Ciliary neurotrophic factor (CNTF), leukemia inhibitory factor (LIF), interleukin-6 (IL-6), prolactin, growth hormone, leptin, interferons (i.e., interferon-α, -β, and -γ), oncostatin M (OSM), Glial cell line-derived neurotrophic factor (GDNF), Artemin, Neurturin, Persephin, and Ephrins; and
a pharmaceutically acceptable carrier.

In some embodiments, the nanoparticles can have an average diameter of about 200 nm to about 800 nm or about 200-500 nm. The dextran sulfate and chitosan can be present in a weight ratio of about 10:1 to about 1:10, preferably about 5:1 to about 1:1, more preferably about 4:1.

In some embodiments, the neurotrophic factor is incorporated into the nanoparticles. The neurotrophic factor can be recombinantly produced using, e.g., recombinant DNA technology known in the art. In certain embodiments, the neurotrophic factor can be CNTF and/or OSM. In one embodiments, the neurotrophic factor is CNTF. In another embodiment, the neurotrophic factor is OSM.

In various embodiments, the nanoparticles can provide sustained and/or prolonged delivery of the neurotrophic factor, e.g., for at least 6 hours, at least 12 hours, at least 24 hours, at least 48 hours, or longer, or any time period therebetween.

In various embodiments, the disease or condition can be a neurodegenerative disease. In some embodiments, the neurodegenerative disease is a retinal neurodegeneration disease. In certain embodiments, the retinal neurodegeneration disease can include retinal degeneration and/or dystrophy (such as Leber congenital amaurosis, retinitis pigmentosa, cone-rod dystrophy, microphthalmia, anophthalmia, myopia, and hyperopia) or retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death).

In certain embodiments, the neurodegenerative disease is selected from schizophrenia, major depression, bipolar disorder, epilepsy, traumatic brain injury, post-traumatic stress disorder, Parkinson's disease, Alzheimer's disease, Down syndrome, spinocerebellar ataxia, amyotrophic lateral sclerosis, Huntington's disease, stroke, radiation therapy induced neuropathy, chemotherapy induced neuropathy, chronic stress, abuse of a neuro-active drug, concussive injury, crush injury, peripheral neuropathy, diabetic neuropathy, post-traumatic headache, multiple sclerosis, spinal cord injury, traumatic spinal cord injury, peripheral nerve injury (such as peripheral nerve crush injury, neonatal brachial plexus palsy, and traumatic facial nerve palsy), Autism, Stargardt disease, Kearns-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.

The present disclosure also provides a method for treating an eye disease of a subject, wherein the method comprises administering to an eye of the subject a therapeutically effective amount of the composition of the present disclosure for treating the eye disease, said eye disease involving a retinal degeneration disorder. The subject may be a mammal. The subject may be a human being. The method achieves a sustained release and a long residing time of the composition.

In one embodiment, the composition can be an ophthalmic formulation such as an eye drop that includes an emulsion. The emulsion can include the nanoparticles dispersed in a liquid. Administering the ophthalmic formulation to an eye of the patient can include applying the eye drop to the eye of the patient.

### BRIEF DESCRIPTION OF THE DRAWINGS

FIG. 1: CNTF and OSM treatment do not have a proliferative effect on TF-1 cells. (A) Bright-field image of TF-1 cells grown in suspension for 3 days. Scale bar=100 μm. (B) Quantitative analysis for the exposure of TF-1 cells supplemented with 1 ng/ml GM-CSF to 20 ng/ml CNTF and 20 ng/ml OSM compared to no treatment control, showing no significant (ns) difference between all groups. (C) Quantitative analysis for the exposure of TF-1 cells supplemented with 0.02 ng/ml GM-CSF and 0.05 ng/ml GM-CSF to increasing concentrations of CNTF (ranging from 20 ng/ml to 180 ng/ml) compared to no GM-CSF treatment control group. Results show that although supplementation with GM-CSF causes an increase in the cell number, treatment with increasing doses of CNTF does not cause a significant effect within each GM-CSF treatment group. Results were reported as Mean±SEM of 3 different cell counts, tested for significant differences via one-way ANOVA analysis with a Tukey's posthoc multiple comparison test (ns=not significant).

FIG. 2: Generation of Eye Field Progenitors (EFPs) and Retinal Neural Progenitors (RNPs) following a multiple-step commitment from human iPSCs. (A) Representative bright-field images of a human iPSCs colonies used as a starting material for directed differentiation into anterior neuroectoderm and EFPs at Day 14 (D14) and RNPs at D21-D35 of differentiation. Cells were induced to differentiate as a monolayer, with subsequent neurosphere formation and attachment for RNPs differentiation program. Scale bar=100 μm. (B) Immunohistochemical analysis at D14 of differentiation showing the high co-expression of RX1 and PAX6 in virtually all cells, indicative of EFP fate, and at D21 of differentiation showing the co-expression of PAX6 with CHX10 in most of the cells, indicative of RNPs fate. Scale bar=100 μm. (C) Flow cytometry analysis at D14 shows that EFPs completely lost the expression pluripotency marker OCT4, while SOX2 and PAX6 expression was highly elevated (96.9%, and 91.4% respectively). Gating was established based on Isotype control (black line).

FIG. 3: Efficient generation and characterization of early Retinal Photoreceptor progenitors (RPPs) derived from human iPSCs. (A) Immunohistological analysis of cells generated at D60 of differentiation shows the high expression of a typical maker of early RPPs (CRX, green), while the expression of a general proliferating marker (Ki67, red) being almost absent, consistent with RPPs characteristic of postmitotic precursor cells. DAPI (blue) was used to counterstain all cell nuclei. Scale bar=100 μm. (B) Quantitative analysis by flow cytometry analysis showing low expression of general neural markers PAX6 (18%) at D60, while specific markers of RPPs, such as CRX (96.4%), NRL (91.2%), and ThRB2 (91.4%) were highly elevated, suggesting a homogeneous population of RPPs. Gating was established based on secondary antibody control (black line). (C) RT-qPCR analysis comparing quantitative gene expression at D14 (EFP stage) and D60 (early RPP stage). At D14 and D60, there is a complete loss of pluripotency gene marker OCT4. At D14 of differentiation, PAX6, RX1 and CHX10 gene expression was elevated compared at D14 compared to D60, while the expression of RPPs gene markers (NRL and NR2E3) and RPPC-like gene markers (REC and RHOD) was low at D13 and increased at D60, suggesting a commitment to RPP fate at this later stage of differentiation.

FIG. 4: CNTF, NP-CNTF, OSM and NP-OSM show a significant pro-survival and pro-proliferation effect on iPS-RPPs. (A) Representative bright-field image of iPS-RPP cells. Scale bar=100 μm. (B) Quantitative analysis showing the significant increase in the normalized cell counts after exposure of iPS-RPP cells to 10 ng/ml CNTF and 10 ng/ml OSM cells compared to no treatment control. (C) Quantitative analysis showing the normalized cell counts after exposure of RPP cells to 10 ng/ml CNTF, NP-CNTF, OSM, and NP-OSM compared to no treatment control showing the significant increase in the RPP cell number after exposure to both native and modified proteins. Results were reported as Mean±SEM of 3 different cell counts, tested for significant differences via one-way ANOVA analysis with a Tukey's posthoc multiple comparison test (Significant P values: ***P<0.001, **P<0.01 and *P<0.05).

FIG. 5: NP-CNTF and NP-OSM promote the survival and proliferation of iPS-RPP cells. (A) Quantitative analysis showing the significant increase in the normalized cell counts after 30 days exposure of 3 different iPS-RPP cell lines (green, orange and purple) to 10 ng/ml CNTF, NP-CNTF, OSM, and NP-OSM compared to no treatment control. (B) The viability of all 3 different cell lines was also significantly increased after the 30 days exposure to 10 ng/ml of CNTF, NP-CNTF, OSM and NP-OSM. (C) Representative bright field images at D14 and D30 (representing the time of analysis) of iPS-RPP cells after the exposure to 10 ng/ml CNTF, NP-CNTF, OSM, and NP-OSM showing qualitatively a clear increase in the cell number and more mature cellular morphology and network for all four treatment conditions compared to controls. Scale bar=100 μm.

FIG. 6: Generation and Characterization of iPS-RGPs following a step-wise commitment from hiPSCs and the pro-proliferative effect of CNTF, NP-CNTF, OSM and NP-OSM. (A) Immunofluorescence images of iPS-RGPs showing the positive expression of MATH5 and BRN3a at D35 of differentiation, and low expression of PAX6, indicative of RGPs fate. DAPI was used to counterstain cell nuclei and bright-field images illustrate the morphology of RGPs. (B) Flow cytometry analysis at D35 confirming the high expression of MATH5 (93.7%), BRN3a (5.8%), and ISL1 (95.9%), associated with low expression of PAX6 (13%) in iPS-RGPs. Gating was established based on secondary antibody control. (C) qPCR analysis demonstrating the elevated temporal gene expression pattern for RGPs markers, MATH5, BRN3a, BRN3b, and ISl1 at D35 of differentiation, associated with no expression at D13 of differentiation. (D) Representative image of iPS-RGPs cells showing the elaborate neuronal network established in vitro. (E) Quantitative analysis showing the normalized cell numbers after the exposure of RGPs to increasing concentration of CNTF, NP-CNTF, OSM and NP-OSM (10, 20, and 40 ng/ml) compared to no treatment control group. All proteins show significant increase in the normalized cell number at all concentration tested, illustrating their pro-proliferative effect in vitro.

FIG. 7: Assessment of spatial visual acuity in both NP-CNTF and NP-OSM-treated rats. (A) Illustration of optokinetic response (OKR) measurement in RCS rats; (B) OKR in units of cycle/degree (c/d) at P60; (C) OKR in units of cycle/degree (c/d) at P90.

FIG. 8: Electroretinography (ERG) measurement of retinal electrical responses to light stimulation in NP-OSM and NP-CNTF treated rats. (A) quantitative comparison of b-wave amplitude of ERG at day P60 in NP-CNTF/OSM treated, sham (BSS) or untreated eyes; (B) P60 ERG of NP-OSM treated eye (red line) and contralateral untreated eye (blue line); (C) P60 ERG of P60 ERG of NP-CNTF treated eye (red line) and contralateral untreated eye (blue line); (D) quantitative comparison of b-wave amplitude of ERG at day P90 in NP-CNTF/OSM treated, sham (BSS) or untreated eyes; (E) P90 ERG of NP-OSM treated eye (red line) and contralateral untreated eye (blue line); (F) P90 ERG of P60 ERG of NP-CNTF treated eye (red line) and contralateral untreated eye (blue line).

FIG. 9: Retinal morphology of NP-OSM treated rat at P90 display remarkable rescue of photoreceptor degeneration. (A) retina from sham treated eye with significant degeneration of outer nuclear layer (ONL); (B) retina from NP-CNTF treated eye showing marginal rescue effect on ONL; (C) retina from NP-OSM treated eye with no ONL degeneration. Magnification: ×200.

FIG. 10: Morphological analysis of NP-OSM treated RCS rat at P90 (A) Immuoflourescence staining of Recoverin (red) in the photoreceptor layer and RPE65 (green) Retinal Pigmented Epithelium (RPE) layer (magnification×200); (B) cross section of the whole retina displaying global preservation of ONL layer. (magnification×25).

FIG. 11: Retinal whole mounts stained with RGC specific marker Brn3 (panel A) and RGC axon specific marker RT97 (panel B) of wild type eyes and ONC eyes treated with different nanoparticles.

FIG. 12: Protection of RGCs in ONC model by NP-CNTF and CN-OSM.

FIGS. 13A-13B: Significant preservation of ERG in both NP-CNTF and NP-OSM treated eyes as compared to eyes injected with control NPs.

FIGS. 14A-14C: Histological analyses showed a broad retinal neural cell preservation in all NP-CNTF and NP-OSM treated eyes of P23H rats, dramatically different from NP treated eyes.

### DETAILED DESCRIPTION

Provided herein, in one aspect, is a composition that achieves sustainable delivery of various neurotrophic factors. A biocompatible nanoparticle (e.g., glycan nanoparticle) system can be adapted. For example, dextran sulfate (DS)-chitosan (CS) polyelectrolyte complexes can be formed into nanoparticles that contain a hydrophobic core and a negatively charged outer shell. Neurotrophic factors can be prepared using recombinant protein technology and incorporated on the outer shell of the nanoparticles at high efficiency. Such compositions can achieve greater in vitro stability and in vivo half-life compared to delivery of the ‘naked’ protein alone, without the nanoparticles. It has been found that various neurotrophic factors, including CNTF and OSM, can be propagated with these biocompatible nanoparticles to form, e.g., nanoparticle-CNTF (NP-CNTF) and NP-OSM, with a desirable in vitro stability profile.

The compositions containing suitable therapeutic concentrations can be administered to mammals by a parenteral route or by topical application, thereby achieving biologically active levels of the active ingredient (e.g., CNTF and/or OSM) longer (e.g., 2-500 times longer) than in a standard vehicle. Further, the active ingredient retains biologic activity over the entire course of release of, e.g., for weeks or months.

### Definitions

For convenience, certain terms employed in the specification, examples, and appended claims are collected here. Unless defined otherwise, all technical and scientific terms used herein have the same meaning as commonly understood by one of ordinary skill in the art to which this disclosure belongs.

The articles “a” and “an” are used herein to refer to one or to more than one (i.e., at least one) of the grammatical object of the article. By way of example, “an element” means one element or more than one element.

As used herein, the term “about” means within 20%, more preferably within 10% and most preferably within 5%. The term “substantially” means more than 50%, preferably more than 80%, and most preferably more than 90% or 95%.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer there between.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, “administering” and similar terms mean delivering, e.g., via intraocular or topical route, the composition to an individual being treated.

As used herein, “biocompatible” refers to compositions that are compatible with and/or not harmful to a living organism. In some embodiments, a biocompatible composition is made of materials that cause no or little adverse effect on the organism. In some embodiments, the intermediates or precursors of the biocompatible composition are compatible with and/or not harmful to the organism. The biocompatible composition can be biodegradable and the end products, e.g., as a result of solubilization or hydrolysis under physiological conditions, or due to digestion by an enzyme or other biological reactions, are compatible with and/or not harmful to the organism. As used herein, “biodegradable” means that the nanoparticles can break down or degrade within the body to non-toxic components.

The term “nanoparticles” as used herein, refers to discrete solid phases having an average diameter of less than 1000 nanometers (nm). Such solid phases can be of any shape. In some embodiments some or all nanoparticles are substantially spherical. Any of a variety of materials can be used to forn or provide particles, as will be understood by those of skill in the art. In some: embodiments, particular materials and/or shapes may be preferred based on chemistries or other features utilized in relevant embodiments; those of ordinary skill will be well familiar with various options and parameters guiding selection. In many embodiments, suitable materials include, but are not limited to, plastics, ceramics, glass, polystyrene, methylstyrene, acrylic polymers, metal, paramagnetic materials, thoria sol, graphitic carbon, titanium dioxide, latex or cross-linked dextrans such as Sepharose, cellulose, nylon, cross-linked micelles and teflon. In some embodiments, the nanoparticles are biocompatible.

In some embodiments, the nanoparticles can be any nanoparticles known in the art, such as those disclosed in PCT International Publication No. WO/2018/026833; Lauten et al., Biomacromolecules. 2010 Jul. 12; 11(7): 1863-1872; Yin et al., Biomacromolecules. 2013 Nov. 11; 14(11): 4009-20; Zaman et al, International Journal of Nanomedicine 2016:11 6149-6159, each of which incorporated herein by reference in its entirety. In some embodiments, the nanoparticles can be dextran sulfate (DS)-chitosan (CS) nanoparticles (DSCS NPs).

In some embodiments, the nanoparticles can be modified to include or incorporate proteins such that the protein can be associated with the nanoparticles via, e.g., electrostatic interactions. The size of protein-incorporated nanoparticles can be substantially the same as free nanoparticles, e.g., between about 10-1000 nm, about 200-800 nm or about 200-500 nm.

As used herein, an “effective amount” means the amount of an agent that is sufficient to provide the desired local or systemic effect at a reasonable risk/benefit ratio as would attend any medical treatment. This will vary depending on the patient, the disease, the treatment being used, and the nature of the agent.

As used herein, “parenteral” shall mean any route of administration other than the alimentary canal and shall specifically include intravitreal, intramuscular, intraperitoneal, intra-abdominal, intra-articular, subcutaneous, and, to the extent feasible, intravenous.

As used herein, “pharmaceutically acceptable” shall refer to that which is useful in preparing a pharmaceutical composition that is generally safe, non-toxic, and neither biologically nor otherwise undesirable and includes that which is acceptable for veterinary use as well as human pharmaceutical use. Examples of “pharmaceutically acceptable liquid carriers” include water and organic solvents. Preferred pharmaceutically acceptable aqueous liquids include PBS, saline, and dextrose solutions.

As used herein, “peptide”, “polypeptide”, “oligopeptide,” and “protein” shall be used interchangeably when referring to peptide or protein drugs and shall not be limited as to any particular molecular weight, peptide sequence or length, field of bioactivity, diagnostic use, or therapeutic use unless specifically stated.

The term “treatment” or “treating” means administration of a drug for purposes including: (i) preventing the disease or condition, that is, causing the clinical symptoms of the disease or condition not to develop; (ii) inhibiting the disease or condition, that is, arresting the development of clinical symptoms; and/or (iii) relieving the disease or condition, that is, causing the regression of clinical symptoms.

Various aspects of the disclosure are described in further detail below. Additional definitions are set out throughout the specification.

Neurotrophic Factors Recent animal studies have shown that photoreceptor cell replacement and/or prevention of ongoing retinal degeneration are two promising therapeutic strategies. The prevention of retinal degeneration is especially promising, as several clinical trials, using human ESC-derived RPE (Schwartz, Hubschman et al. 2012, Schwartz, Regillo et al. 2015), fetal central neural stem cells (ClinicalTrials.gov, Identifier: NCT01632527; Sponsor: Stem Cell Inc) and fetal photoreceptor progenitors (ClinicalTrials.gov, Identifiers: NCT02320812; Sponsor: jCyte, Inc and NCT02464436; Sponsor: ReNeuron Inc), are currently underway. There is also substantial evidence that exogenous neurotrophic factors, including glial-derived neurotrophic factor (GDNF) (Buch, MacLaren et al. 2006, Del Rio, Irmler et al. 2011), ciliary neurotrophic factor (CNTF) (Sieving, Caruso et al. 2006, Zhang, Hopkins et al. 2011), oncostatin M (OSM) (Xia, Li et al. 2011), brain-derived neurotrophic factor (BDNF) (Azadi, Johnson et al. 2007, Zhou, Kim et al. 2009) and pigment epithelium-derived factor (PEDF) (Jablonski, Tombran-Tink et al. 2000) have powerful neuroprotective effect on host photoreceptors in several animal models.

CNTF and OSM are two potent survival factors for neurons and oligodendrocytes. CNTF has also been shown to prevent the degeneration of motor axons after axotomy. CNTF and OSM are members of IL-6 cytokine family which includes IL6, IL11, IL-27, G-CSF, CLC, CT-1, Neuropoietin, Leptin/OB and LIF (Rose-John, 2018). Li et al (Li, Tao et al. 2010) and Xia et al (Xia, Li et al. 2011) demonstrated that administration of CNTF or OSM not only protected rod and cone photoreceptors from degeneration, but also promoted regeneration of the cone outer segment in RCS and S334-ter transgenic rats. Clinical studies showed that CNTF treatment resulted in a dose-dependent increase in retina thickness and apparent stabilization of visual acuity (Zhang, Hopkins et al. 2011). However, transplantation of cells engineered to express CNTF into the eye leads to some unexpected side effects: secretion of other uncharacterized factors caused inflammatory reactions in the eye. While injection of CNTF and OSM into the vitreous protected photoreceptors from degeneration in animal studies, frequent administration (every 2-3 days) limits the application of this approach in clinics (Li, Tao et al. 2010, Xia, Li et al. 2011).

Provided herein, in some embodiments, is an improved delivery technology which incorporates various neurotrophic factors into dextran sulfate (DS)-chitosan (CS) nanoparticles (DSCS NPs) to achieve sustained release and long residing time of the neurotrophic factors. Without wishing to be bound by theory, it is believed that the neurotrophic factors can be heparin-binding proteins which can bind DSCS NPs via electrostatic interactions. Heparin-binding proteins are defined by their high affinity for heparin, which is commonly demonstrated by binding the proteins to heparin-Sepharose resin in >0.15 M NaCl. The biological ligands of most of these proteins are heparan sulfate or sulfated glycosaminoglycans, which are present on the cell surface and in the extracellular matrix. A heparin-binding site or domain in the primary or tertiary structure of the proteins is responsible for heparin binding, which often includes a cluster of positively charged amino acid residues (Lys, Arg, and His) that bind to heparin through electrostatic interaction. As DS is an analog of heparin, heparin-binding proteins will likely bind to negatively charged DSCS NPs.

Neurotrophic factors (NTFs) are a family of biomolecules—nearly all of which are peptides or small proteins—that support the growth, survival, and differentiation of both developing and mature neurons. Exemplary neurotrophic factors include:
1. Neurotrophins (Budni, J., T. Bellettini-Santos, F. Mina, M. L. Garcez, and A. I. Zugno. 2015. ‘The involvement of BDNF, NGF and GDNF in aging and Alzheimer's disease’, Aging Dis, 6: 331-41) such as Brain-derived neurotrophic factor (BDNF), Nerve growth factor (NGF), Neurotrophin-3 (NT-3), and Neurotrophin-4 (NT-4).
2. Neuropoietic cytokines (such as CNTF family) (Rose-John, S. 2018. ‘Interleukin-6 Family Cytokines’, Cold Spring Harb Perspect Biol, 10) such as Ciliary neurotrophic factor (CNTF), leukemia inhibitory factor (LIF), interleukin-6 (IL-6), prolactin, growth hormone, leptin, interferons (i.e., interferon-α, -β, and -γ), and oncostatin M.
3. GDNF family (Ibanez, C. F., and J. O. Andressoo. 2017. ‘Biology of GDNF and its receptors—Relevance for disorders of the central nervous system’, Neurobiol Dis, 97: 80-89) such as Glial cell line-derived neurotrophic factor (GDNF), Artemin, Neurturin, Persephin and Ephrins.

Additional neurotrophic factors include: glia maturation factor, insulin, insulin-like growth factor 1 (IGF-1), vascular endothelial growth factor (VEGF), fibroblast growth factor (FGF), platelet-derived growth factor (PDGF), pituitary adenylate cyclase-activating peptide (PACAP), interleukin-1 (IL-1), interleukin-2 (IL-2), interleukin-3 (IL-3), interleukin-5 (IL-5), interleukin-8 (IL-8), macrophage colony-stimulating factor (M-CSF), granulocyte-macrophage colony-stimulating factor (GM-CSF), and neurotactin.

Each family may its own distinct cell signaling mechanisms, although the cellular responses elicited often do overlap.

In some embodiments, the neurotrophic factor can be recombinantly produced using, e.g., recombinant DNA technology known in the art.

### Pharmaceutical Compositions and Use Thereof

It has been shown herein that various neurotrophic factors can be incorporated into dextran sulfate (DS)-chitosan (CS) nanoparticles (DSCS NPs) to achieve sustained release and long residing time. In particular, CNTF and OSM have been tested extensively. As shown herein, TF-1 cells have been derived from a patient with erythroleukaemia and widely used in proliferation bioassays by manufacturers for their routine quality assurance and quality control of multiple cytokines including CNTF and OSM. However, surprisingly, it has been repeatedly observed herein that neither CNTF nor OSM is capable of stimulating TF-1 cell proliferation in vitro with or without GM-CSF. These unexpected observations lead to the speculation that the pro-survival and pro-proliferation effects of CNTF and OSM may be cell type specific. As such, an assay platform has been developed using human pluripotent stem cells (including human embryonic stem cells and induced pluripotent stem cells: hESCs and iPSCs) derived photoreceptor progenitors and retinal ganglion progenitors (iPS-RPPs and iPS-RGPs) to evaluate the biologic functionality of NP-CNTF and NP-OSM in vitro. The results showed that both NP-CNTF and NP-OSM enhanced the survival and proliferation of iPS-RPPs and iPS-RGPs in a wide range of doses, similar to native CNTF and OSM, demonstrating that the appropriate target cells are critical for successful evaluation of potential biological effects in vitro. Furthermore, when administered intravitreally in Royal College of Surgeon (RCS) rats, a single dose of NP-OSM not only halt/delay the decline of visual functions in these animals as compared to control NP treated ones, but also preserved the integrity of retinal structure for an extended period of time, clearly demonstrating neuroprotective function in this genetic macular degeneration model. NP-CNTF also displayed similar effects, but to a lesser extent. These results strongly indicate the therapeutic potentials of the stable and biocompatible NP-OSM and NP-CNTF for retinal neuron degeneration diseases and other neurodegenerative diseases as well.

As such, provided herein are pharmaceutical compositions containing the DSCS NPs with one or more neurotrophic factors incorporated therein and a pharmaceutically acceptable carrier, for sustained release and long residing time of the neurotrophic factors. In some embodiments, the pharmaceutical composition contains the NP-OSM and/or NP-CNTF disclosed herein, and a pharmaceutically acceptable carrier. For example, the nanoparticles used in the present disclosure can be part of a pharmaceutical composition. Said pharmaceutical compositions include any solid, semi-solid or liquid (i.e., suspension or dispersion of the nanoparticles of the present disclosure) composition for application by oral, topical, parenteral or ocular route, or any composition in the form of a gel, ointment, cream or balm for administration by topical or ocular route.

In some embodiments, incorporation of proteins into DSCS NPs can be achieved using, e.g., entrapment procedures, in which protein molecules are mixed with DS and CS until particle formation occurs. Incorporation can also be achieved by mixing proteins with preformed DSCS NPs and allowing binding of the protein to the NPs. In some embodiments, the proteins can bind to the outer shell of the NPs via electrostatic interactions.

The size of successfully incorporated protein nanoparticles are between about 10-1000 nm, about 200-800 nm or about 200-500 nm. The protein:DS:CS mixing ratio can be about (0.1-0.5):(1-3):(0.15-0.5). Exemplary methods are disclosed in Lauten et al., Biomacromolecules. 2010 Jul. 12; 11(7): 1863-1872; Yin et al., Biomacromolecules. 2013 Nov. 11; 14(11): 4009-20; Zaman et al, International Journal of Nanomedicine 2016:11 6149-6159, each of which incorporated herein by reference in its entirety.

The term “pharmaceutically acceptable carrier” refers to a carrier or adjuvant that may be administered to a subject (e.g., a patient), together with a nanoparticle composition of the present disclosure, and which does not destroy the pharmacological activity thereof and is nontoxic when administered in doses sufficient to deliver a therapeutic amount of the nanoparticle composition.

Pharmaceutically acceptable carriers, adjuvants and vehicles that may be used in the compositions of the present disclosure include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts, or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of nanoparticle compositions described herein.

The compositions for administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, losenges or the like in the case of solid compositions. In such compositions, the nanoparticle composition is usually a minor component (from about 0.1 to about 50% by weight or preferably from about 1 to about 40% by weight) with the remainder being various vehicles or carriers and processing aids helpful for forming the desired dosing form.

The amount administered depends on the nanoparticle composition formulation, route of administration, etc. and is generally empirically determined in routine trials, and variations will necessarily occur depending on the target, the host, and the route of administration, etc. Generally, the quantity of active nanoparticle composition in a unit dose of preparation may be varied or adjusted from about 1, 3, 10 or 30 to about 30, 100, 300 or 1000 mg, according to the particular application. In a particular embodiment, unit dosage forms are packaged in a multipack adapted for sequential use, such as blisterpack, comprising sheets of at least 6, 9 or 12 unit dosage forms. The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the nanoparticle composition. Thereafter, the dosage is increased by small amounts until the optimum effect under the circumstances is reached. For convenience, the total daily dosage may be divided and administered in portions during the day if desired.

In one aspect, methods for treating (e.g., controlling, relieving, ameliorating, alleviating, or slowing the progression of) or methods for preventing (e.g., delaying the onset of or reducing the risk of developing) one or more diseases, disorders, or conditions caused by, or associated with, accelerated neuron cell death and/or axonal degeneration in a subject in need thereof are featured. The methods include administering to the subject an effective amount of a nanoparticle composition as defined anywhere herein to the subject.

In another aspect, the use of a nanoparticle composition as defined anywhere herein in the preparation of, or for use as, a medicament for the treatment (e.g., controlling, relieving, ameliorating, alleviating, or slowing the progression of) or prevention (e.g., delaying the onset of or reducing the risk of developing) of one or more diseases, disorders, or conditions caused by, or associated with, exacerbated neuronal cell death and/or axonal degeneration is featured.

In embodiments, the one or more diseases, disorders, or conditions can include neuropathies, nerve trauma, and neurodegenerative diseases. In embodiments, the one or more diseases, disorders, or conditions can be diseases, disorders, or conditions caused by, or associated with accelerated death of existing neurons and/or axonal degeneration. Examples of the one or more diseases include, but are not limited to, schizophrenia, major depression, bipolar disorder, epilepsy, traumatic brain injury and/or a visual symptom associated therewith, post-traumatic stress disorder, Parkinson's disease, Alzheimer's disease, Down syndrome, spinocerebellar ataxia, amyotrophic lateral sclerosis, Huntington's disease, stroke, radiation therapy induced neuropathy, chemotherapy induced neuropathy, chronic stress, abuse of a neuro-active drug, concussive injury, crush injury, peripheral neuropathy, diabetic neuropathy, post-traumatic headache, multiple sclerosis, retinal degeneration and dystrophy (such as Leber congenital amaurosis, retinitis pigmentosa, cone-rod dystrophy, microphthalmia, anophthalmia, myopia, and hyperopia), retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death), spinal cord injury, traumatic spinal cord injury, peripheral nerve injury (such as peripheral nerve crush injury, neonatal brachial plexus palsy, and traumatic facial nerve palsy), Autism, Stargardt disease, Keams-Sayre syndrome, Pure neurosensory deafness, Hereditary hearing loss with retinal diseases, Hereditary hearing loss with system atrophies of the nervous system, Progressive spinal muscular atrophy, Progressive bulbar palsy, Primary lateral sclerosis, Hereditary forms of progressive muscular atrophy and spastic paraplegia, Frontotemporal dementia, Dementia with Lewy bodies, Corticobasal degeneration, Progressive supranuclear palsy, Prion disorders causing neurodegeneration, Multiple system atrophy (olivopontocerebellar atrophy), Hereditary spastic paraparesis, Friedreich ataxia, Non-Friedreich ataxia, Spinocerebellar atrophies, Amyloidoses, Metabolic-related (e.g., Diabetes) neurodegenerative disorders, Toxin-related neurodegenerative disorders, Charcot Marie Tooth, Diabetic neuropathy, Metabolic neuropathies, Endocrine neuropathies, Orthostatic hypotension, Creutzfeldt-Jacob Disease, Primary progressive aphasia, Frontotemporal Lobar Degeneration, Cortical blindness, Shy-Drager Syndrome (Multiple, System Atrophy with Orthostatic Hypotension), Diffuse cerebral cortical atrophy of non-Alzheimer type, Lewy-body dementia, Pick disease (lobar atrophy), Thalamic degeneration, Mesolimbocortical dementia of non-Alzheimer type, Nonhuntingtonian types of chorea and dementia, Cortical-striatal-spinal degeneration, Dementia-Parkinson-amyotrophic lateral sclerosis complex, Cerebrocerebellar degeneration, Cortico-basal ganglionic degeneration, Familial dementia with spastic paraparesis or myoclonus, and Tourette syndrome.

Suitable assays which directly or indirectly detect neural survival, growth, development, function and/or generation are known in the art, including axon regeneration in rat models (e.g. Park et al., Science. 2008 Nov. 7; 322:963-6), nerve regeneration in a rabbit facial nerve injury models (e.g. Zhang et al., J Transl Med. 2008 Nov. 5; 6(1):67); sciatic nerve regeneration in rat models (e.g. Sun et al., Cell Mol Neurobiol. 2008 Nov. 6); protection against motor neuron degeneration in mice (e.g. Poesen et al., J. Neurosci. 2008 Oct. 15; 28(42):10451-9); rat model of Alzheimer's disease, (e.g. Xuan et al., Neurosci Lett. 2008 Aug. 8; 440(3):331-5); animal models of depression (e.g. Schmidt et al., Behav Pharmacol. 2007 Sep.; 18(5-6):391-418; Krishnan et al., Nature 2008, 455, 894-902); and/or those exemplified herein.

The nanoparticle compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/kg to about 1000 mg/kg, (e.g., from about 0.01 to about 100 mg/kg, from about 0.1 to about 100 mg/kg, from about 1 to about 100 mg/kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, New York, 537 (1970). In certain embodiments, the compositions are administered by oral administration or administration by injection. The methods herein contemplate administration of an effective amount of nanoparticle composition to achieve the desired or stated effect. Typically, the pharmaceutical compositions of the present disclosure will be administered from about 1 to about 6 times per day or alternatively, as a continuous infusion. Such administration can be used as a chronic or acute therapy.

According to a particular embodiment, the nanoparticles of the present disclosure or the pharmaceutical composition comprising them is administered by ocular route, preferably by means of subretinal injection, intravitreal injection, subconjunctival injection or by means of topical administration in the eye.

Lower or higher doses than those recited above may be required. Specific dosage and treatment regimens for any particular patient will depend upon a variety of factors, including the activity of the specific nanoparticle composition employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a nanoparticle composition of the present disclosure may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

In some embodiments, the nanoparticle compositions described herein can be co-administered with one or more other therapeutic agents. In certain embodiments, the additional agents may be administered separately, as part of a multiple dose regimen, from the nanoparticle compositions of the present disclosure (e.g., sequentially, e.g., on different overlapping schedules with the administration of one or more nanoparticle compositions. In other embodiments, these agents may be part of a single dosage form, mixed together with the nanoparticle compositions of the present disclosure in a single composition. In still another embodiment, these agents can be given as a separate dose that is administered at about the same time that one or more nanoparticle compositions are administered (e.g., simultaneously with the administration of one or more nanoparticle composition). When the compositions of the present disclosure include a combination of a nanoparticle composition described herein and one or more additional therapeutic or prophylactic agents, both the nanoparticle composition and the additional agent can be present at dosage levels of between about 1 to 100%, and more preferably between about 5 to 95% of the dosage normally administered in a monotherapy regimen.

The compositions of the present disclosure may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated nanoparticle composition or its delivery form.

The pharmaceutical compositions can further comprise pH controlling agents, such as, for example, buffer agents preventing the pH of the composition from dropping to values below 5, antioxidant agents inhibiting oxidation of the lipid component, as well as preservatives preventing significant structural changes in the formulation. Depending on their function, these additional components can be present in the phases making up the nanoparticles or in the aqueous medium where they are dispersed or completely or partially adsorbed thereon. The person skilled in the art can determine what additional components can be used and if they are necessary, many of them being commonly used in pharmaceutical and cosmetic compositions.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of the present disclosure may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of the present disclosure may also be administered in the form of suppositories for rectal administration. These compositions can be prepared by mixing a nanoparticle composition of the present disclosure with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of the present disclosure is useful when the desired treatment involves areas or organs readily accessible by topical application. For application topically to the skin, the composition should be formulated with a suitable ointment containing the active components suspended or dissolved in a carrier. Carriers for topical administration of the nanoparticle compositions of the present disclosure include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active nanoparticle composition suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of the present disclosure may also be topically applied to the lower intestinal tract by rectal suppository formulation or in a suitable enema formulation.

In some embodiments, topical administration of the compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Remington's Pharmaceutical Sciences, 21st Edition (2005) published by Mack Publishing Company, which is incorporated herein by reference in its entirety.

Topically-transdermal patches are also included in the present disclosure. Also within the present disclosure is a patch to deliver active chemotherapeutic combinations herein. A patch includes a material layer (e.g., polymeric, cloth, gauze, bandage) and the nanoparticle compositions herein as delineated herein. One side of the material layer can have a protective layer adhered to it to resist passage of the compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of the present disclosure may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the nanoparticles herein and an additional agent (e.g., a therapeutic agent) can be administered using any of the routes of administration described herein. In some embodiments, a composition having nanoparticles herein and an additional agent (e.g., a therapeutic agent) can be administered using an implantable device.

Implantable devices and related technology are known in the art and are useful as delivery systems where a continuous, or timed-release delivery of compositions delineated herein is desired. Additionally, the implantable device delivery system is useful for targeting specific points of composition delivery (e.g., localized sites, organs). Negrin et al., Biomaterials, 22(6):563 (2001). Timed-release technology involving alternate delivery methods can also be used in the present disclosure. For example, timed-release formulations based on polymer technologies, sustained-release techniques and encapsulation techniques (e.g., polymeric, liposomal) can also be used for delivery of the compositions delineated herein.

The present disclosure will be further described in the following examples. It should be understood that these examples are for illustrative purposes only and are not to be construed as limiting the present disclosure in any manner.

### EXAMPLES

### Example 1: Neuroprotective Function of Biocompatible Nanoparticle-CNTF/OSM In Vitro and in Retinal Degenerative Rats

Retinal degeneration is an irreversible process that ultimately leads to untreatable blindness and prevention of ongoing retinal degeneration is a promising therapeutic strategy. Ciliary neurotrophic factor (CNTF) and Oncostatin M (OSM) are two potent survival factors for neurons and oligodendrocytes. Stable and biocompatible nanoparticles incorporated with CNTF and OSM (NP-CNTF and NP-OSM) were produced. To evaluate their neuroprotective effects, novel in vitro assay platforms using retinal photoreceptor progenitor (RPP) and retinal ganglion progenitors (RGP) cells derived from human induced pluripotent stem cells (hiPSC) was developed. Significant pro-survival and pro-proliferation effects of both NP-CNTF and NP-OSM were observed in both iPS-RPP and iPS-RGP platforms within a broad range of doses (from 0.2 to 20 ng/ml). No such effect was observed in an identical assay using TF-1 cells that have been widely used in proliferation bioassays by manufacturers for routine quality assurance and quality control of multiple cytokines including CNTF and OSM, suggesting the pro-survival and pro-proliferation effects of NP-CNTF and NP-OSM are neural lineage specific.

To investigate their neuroprotective effects in vivo, a single dose of NP-CNTF, NP-OSM, or control NPs were injected intravitreally in Royal College of Surgeon (RCS) rats, and electroretinography (ERG) and optical kinetic response (OKR) were carried out at postnatal day 60 and 90 (P60 and P90). Significant preservation of ERG and OKR in both NP-CNTF and NP-OSM treated animals as compared to rats injected with NPs and untreated controls was detected, and a more profound effect of NP-OSM was observed. Histological analyses showed a global retinal neural cell preservation in NP-OSM treated RCS rats, dramatically different from NP-CNTF and NP treated and untreated control animals at P90. These results demonstrate that stable and biocompatible NP-OSM, and NP-CNTF in a less degree, are neuroprotective both in vitro and in a genetic retinal degeneration animal model, indicating a strong therapeutic value for retinal neurodegeneration diseases and other neurodegenerative diseases as well.

### Materials & Methods

Incorporating Recombinant Proteins into Nanoparticles: In order to incorporate therapeutic proteins into DSCS NPs efficiently, we choose heparin-binding proteins for the incorporation, as DS in the outer shell of DSCS NPs is an analog of heparin. An Azure A assay was established to quantify charged DS in the particles; corresponding glucose sulfate units in the DS were calculated to estimate the loading capacity of DSCS NPs. Previous data show that incorporation efficiencies of heparin-binding proteins (SDF-1α, VEGF, FGF-2, BMP-2, or lysozyme) to preformed DSCS NPs were between 95% and 100%. The size and zeta potential of the NPs were not significantly affected at protein loading conditions up to ˜1.5 nmol of monomeric or ˜0.75 nmol of dimeric heparin-binding proteins per 100 nmol charged glucose sulfate units in DSCS NPs. In comparison, nonspecific adsorption of BSA or gamma globulin to DSCS NPs showed minimal incorporation in 50% PBS (8% and 4%, respectively) but a substantial incorporation in water (80% and 92%, respectively). Incorporation into DSCS NPs did not interfere with the activities of SDF-1α and VEGF, and enabled greater thermal stability of the proteins. It also markedly extended the in vivo retention time of SDF-1α compared to its free form. These data show that heparin binding domain-mediated specific interaction allows an efficient incorporation of proteins into preformed DSCS NPs. This incorporation provides enhanced in vitro and in vivo stability of the proteins, which could be useful for their various biological applications.

Prior to protein incorporation, aliquots of lyophilized DSCS NPs were reconstituted with water and centrifuged at 20,000×g for 15 min to remove ultrafine particles. Pellets were resuspended in 2.5% mannitol, and an Azure A assay was performed to determine the amount of charged DS in the NPs. (Ultrafine particles could have contributed to 10%-15% of charged DS in the lyophilized particles. Thus, it was necessary to confirm the charged DS content with the Azure A assay after centrifugation.) Incorporation reactions were carried out by diluting specified amounts of DSCS NP and protein in water, 50% phosphate-buffered saline (PBS), or otherwise noted buffer solutions, and adding protein solution slowly to NPs while stirring at 800 rpm. Total reaction volume was 0.3 mL or in some cases 0.15 mL which were placed in a 2 mL glass vial with a 1.5×8 mm² stir bar, or a 1.5 mL tube with a 3×3 mm² stir bar, respectively. The mixture was stirred at 300 rpm for another 20 min. After the incorporation reactions, NPs were separated from unincorporated protein by centrifugation at 21,000×g for 20 min. Supernatants were collected and pellets were resuspended in 2.5% mannitol to their original volume. Equal volumes of supernatants and pellets were loaded on a 4%-20% sodium dodecyl sulfate (SDS) gel for electrophoresis. Gels were stained with Coomassie blue, and protein bands were quantified by densitometric analysis using BioRad ImageLab.

All NP-OSM and NP-CNTF used in this study were produced following the established method as described above.

### TF-1 Cells Culture:

TF-1 cells (factor-dependent human erythroleukemic cell line) (ATCC CRL-2003™) were routinely expanded as single cells in suspension (at the recommended density of 3 to 5×10⁴ viable cells/ml) in RPMI-1640 complete media consisting of RPMI-1640 (ThermoFisher Scientific) supplemented with 1 ng/ml recombinant human GM-CSF (granulocyte-macrophage colony-stimulating factor) (Novoprotein) and 10% Fetal Bovine Serum (FBS) (ThermoFisher Scientific). Cells were expanded twice after thawing at 2-3 days intervals, followed by different treatment protocols. Briefly, cell suspension was collected and centrifuged at 300 g for 3 min, and cell pellet re-suspended with fresh medium with and without the different proteins and plated as necessary. To determine the long-term growth effect of GM-CSF on TF1 cells upon treatment, cells were plated in low adhesion 24-well plates at 8-16×10¹ cells/well in fresh RPMI complete medium supplemented with different concentration of GM-CSF (1 ng/ml, 0.05 ng/ml, and 0.02 ng/ml. Cultures were maintained in suspension at 37° C. in a humidified atmosphere of 95% air/5% CO2.

RPPs and RGPs Differentiation from Human iPSCs

Three induced pluripotent stem cell (iPSC) lines used in this study were generated from human normal dermal fibroblast (hNDF) cells by using the StemRNA™-NM Reprogramming kit (Stemgent, Cat #00-0076). iPSCs were grown in vitro as colonies on 0.25 μg/cm² iMatrix-511 Stem Cell Culture Substrate (Recombinant Laminin-511) (ReproCell) and cultured in NutriStem XF/FF™ Culture (Biological Industries) for at least 3 passages prior to directed differentiation into retinal lineage. iPSCs were passaged as cell clumps using Versene (ThermoFisher Scientific). iPSCs were induced to directly differentiate into retinal Photoreceptor progenitor (RPP) lineage following a newly established protocols. Briefly, neural induction was initiated from iPSCs colonies at around 35% confluency as a monolayer by culturing in NIM (Neural Induction Medium) consisting of DMEM/F12 with Hepes, 1% N2 and 1% B27 serum-free supplements, 1% penicillin/streptomycin, MEM Non-essential amino acids (ThermoFisher Scientific), 0.30% glucose (Sigma), 20 μg/ml human insulin (Roche) and 100 ng/ml human Noggin (Peprotech). Cells were incubated at 37° C. in humidified 5% CO₂/95% air atmosphere. The medium was changed daily. At around day 14 (D14), when cells reached 100% confluency and expressed more than 95% of PAX6 by flow cytometry, cells were manually lifted with a scraper (Corning) and plated into ultra-low attachment dishes in NDM (Neural Differentiation Media) consisting of NIM without Noggin and Insulin. Subsequently, the formed neurospheres grown in suspension were attached after 5 days on Laminin-511 coated dishes. Cells were maintained in NDM until about day 60 (D60) with half medium change every 2-3 days. Cell were then manually scraped and plated on ultra-low attachment dishes at 1:1 seeding density to form neurospheres in suspension for 2-3 days. Cells were cryopreserved as neurospheres in Cryostem freezing media (Biological Industries) and banked for future experimental assays. For the current study, neurospheres were repeatedly thawed in NDM media, dissociated with TripLE (ThermoFisher Scientific) into single cells following the manufacturer's instructions, resuspended in NDM and seeded on Matrigel (Corning) coated 24-well plates at a density of 5-10×10⁴ cells/well. Treatment was initiated 48 hours post seeding.

The induction of hiPSCs towards retinal ganglion progenitors (RGPs) employed a procedure similar to that of generation of iPS-RPPs for the initial stages up to D13 of differentiation. Briefly, iPSCs colonies at 35% confluency were cultured in NIM and incubated at 37° C. in humidified 5% CO2/95% air atmosphere with medium changed daily. At around D13, when cells reached 100% confluency and expressed more than 95% of PAX6 by flow cytometry, culture medium was changed to RGP (Neural Ganglion Medium), where DMEM/F12 in NIM media was replaced with Neurobasal medium (ThermoFisher Scientific) supplemented with 5 M Forskolin and 10 ng/ml BDNF to promote neuronal survival. Cultures were continued as a monolayer for another 5-6 days with half media change every 2 days. At about 20 days in cultures, cells were manually scraped into fragments in RGP medium and transferred into low-attachment dishes to form neurospheres in suspension for another 3-5 days. Neurospheres were attached onto Matrigel coated dishes in RGP medium and maintained for another 14 days until they reached the early RGP stage at around D35 in vitro. For the current study, D35 cultures were dissociated with TripLE into single cells following the manufacturer's instructions, resuspended in RGP medium without BDNF, and seeded on Matrigel coated 24-well plates at a density of 5×10⁴ cells/well, followed by treatment with CNTF, NP-CNTF, OSM and NP-OSM cytokines 48 hours post seeding.

Biological Function Assays of CNTF, OSM, NP-CNTF and NP-OSM with TF-1, iPS-RPP and iPS-RGP Cells

CNTF and OSM incorporation into nanoparticles were carried out as described using entrapment procedures as reported earlier, in which protein molecules are mixed with DS and CS until particle formation occurs. (Zaman, Wang et al. 2016)

For TF1 cell suspension treatment, cells were centrifuged and resuspended in fresh media supplemented with 1 ng/ml GM-CSF and CNTF, CNTF-NP, OSM, and CNTF (each at 20 ng/ml). To unmask the proliferative effect of high dose of 1 ng/ml GM-CSF, TF1 cell were treated with CNTF (20 ng/ml-180 ng/ml) in fresh media supplemented with low doses (0.02 ng/ml and 0.05 ng/ml) GM-CSF. Protein treatment was omitted in control groups. The effect of proteins at different GM-CSF concentrations was evaluated by cell counting after the indicated number of days in culture (3-4 days). The number of viable cells and viability (%) was determined using a Cellometer Auto 2000 cell counter (Nexcelom Bioscience) and AOPI staining solution in PBS (Nexcelom Bioscience). Treatments were performed in triplicates and experiments were repeated at least twice.

For iPS-RPP and iPS-RGP cells treatment, cells were treated with carrier-free CNTF and OSM, and NP-CNTF and NP-OSM in NDM 48 hours post attachment, with media change every 3-4 days while maintaining the appropriate concentration of proteins in media. In an additional experiment to determine the optimal concentration of nanoparticle proteins leading to a maximal effect, cells were treated with increasing concentration of NP-CNTF (2 ng/ml to 20 ng/ml), and NP-OSM (2 ng/ml to 20 ng/ml). iPS-RPP and iPS-RGP cells derived from three different iPSC lines were treated with 10 ng/ml NP-CNTF and 10 ng/ml NP-OSM. For comparison purposes, proteins were omitted in control groups. After 4 weeks post treatment, cells were prepared for quantitative analysis by cell counting of viable cells as described previously. Treatments were performed in triplicates and experiments were repeated at least twice.

### Immunocytochemistry:

iPS-RPP or iPS-RGP cells were dissociated into single cells using TripLE and seeded on Matrigel coated 24-well plates in vitro at a 5.0×10⁴ cells/well for 2 days in NDM medium.

Medium was removed, cells washed 3 times with DPBS with Ca/Mg (ThermoFisher Scientific), and then fixed with 4% PFA (paraformaldehyde) (Electron Microscopy Sciences) for 15 minutes at room temperature followed by washing 3 times with DPBS. Cells were permeabilized and blocked with 5% normal donkey serum (NDS) (Jackson Immunolab) and 0.1% Triton X-100 (Sigma) in DPBS at room temperature for up to 1 hour, followed by incubation with primary antibodies diluted in blocking buffer overnight at 4° C. Primary antibodies and their dilution and source used for staining are summarized in Table 1. After overnight antibody incubation, the cells were washed 3 times with DPBS followed by subsequent incubation for 2 hours at room temperature under dark condition with the appropriate species specific fluorescently conjugated secondary antibodies diluted in DPBS containing 2.5% NDS and 0.1% Triton X-100: donkey anti-mouse Alexa Fluor® 488-[1:1000] and donkey anti-rabbit Alexa Fluor® 594-conjugated secondary antibodies [1:1000](ThermoFisher Scientific). Secondary antibodies used for immunostaining are listed in Table2. Cells were washed 3 times with DPBS and cell nuclei were counterstained with 1 μg/ml 4′,6-diamidino-2-phenylindole, dihydrochloride (DAPI) (Thermo Scientific, Cat #62247) for 3 minutes at room temperature, followed by DPBS washing. Cells were examined using a computer-assisted Nikon inverted microscope (Eclipse Ti-S) with a 10× and 20×objective, and images were captured and analyzed using NIS-Elements-BR software (Version 4.50, Nikon).

### Flow Cytometry Analysis:

Cultures was dissociated into single cells with TrypLE (ThermoFisher Scientific), filtered through 40 m strainer, and fixed with Fixation/Permeabilization buffer (BD Biosciences) for 12 minutes on ice. For flow cytometry using fluorescently conjugated antibodies to detect intracellular antigens, fixed 1.0-2.0×10⁵ cells/tube were permeabilized with ice-cold 1×BD Perm/Wash Buffer containing FBS and Saponin (BD Biosciences, Cat #554714) for 30 minutes on ice, followed by incubation with appropriately conjugated antibodies (Summarized in Table 1) for 30 minutes under dark conditions. Cells were than washed with 2 ml of Perm/Wash buffer and prepared for analysis. Control cells were incubated with mouse or rabbit IgG. For flow cytometry using unconjugated antibodies to detect intracellular antigens, fixed cells were blocked with blocking buffer consisting of 0.05% Triton X-100 (Sigma) and 5% normal donkey serum (NDS) (Jackson ImmunoResearch) in DPBS (Life Technologies) (Life technology, Cat #14190250) for 30 minutes on ice, followed by incubation with primary antibodies diluted in blocking buffer for 1 hour at room temperature, then washed with blocking buffer. Cells were then incubated with the appropriate donkey anti-rabbit Alexa Fluor® 488 (Invitrogen, Cat #A21206) or donkey anti-mouse Alexa Fluor®647-conjugated secondary antibodies (Invitrogen, Cat #A31571) diluted in blocking buffer [1:1000] for 1 hour under dark conditions. After washing, cells were resuspended in blocking buffer. Control cells were incubated in secondary antibodies. Cells were analysed on an Accuri C6 flow cytometer (BD Biosciences) according to standard procedures. Data were analyzed with the X software (BD).

Quantitative Real-Time Polymerase Chain Reaction (qPCR):

Total RNA was isolated from cultured cells using the RNeasy Minikit (Qiagen), and concentration was measured using NanoDrop One (Thermo Scientific). qPCR was performed in a two-step reaction. For reverse transcription (RT), 0.5 micrograms of total RNA were transcribed to cDNA using the SuperScript OSM-IV VILO Master Mix cDNA Synthesis kit (Invitrogen) in accordance with the manufacturer's instructions, using a SympliAmp Thermal Cycler (Applied Biosystems). For qPCR reactions, cDNA was amplified in 20xl reaction mixtures containing TaqMan Gene Expression Assays and TaqMan Fast Advanced Master Mix (Applied Biosystems) using the QuantStudio™ 6 Flex Real-Time PCR System with 96-well plate block. All TaqMan Gene Expression Assays (TaqMan probes) used for the experiments are listed in Table 3. In this study, the house-keeping gene GAPDH was used as internal control for data normalization. Relative quantification data for each target gene was analyzed using the QuantStudio Real-Time PCR v1.3 software, based on 2^{(-ΔΔCT)} method (Livak and Schmittgen 2001), with induced pluripotent stem cells (iPSCs) used as the reference control. Samples were done in triplicates and collected from 3 independent cultures.

### Intravitreal Injection of NP-CNTF, NP-OSM and Control NP:

Pigmented dystrophic RCS rats at postnatal day 20 (P20) were used for the current study. A suspension containing equivalent of 2 ug CNTF or OSM in 2 μl balance salt solution (BSS) were injected into the vitreous through a 33-gauge needle connected to a to a 25 μl syringe (Hamilton, Reno, NV). Rats were received unilateral intravitreal injections of NP-CNTF or NP-OSM, the fellow eyes served as sham-operated with NP injection or untreated controls.

### Assessment of Visual Function:

Visual function were tested at 2 time points (postnatal Day 60 and 90)) after injection by Optokinetic response (OKR) and electroretinography (ERG).

Optokinetic response (OKR): Visual acuity were tested by OKR using an Optomotry testing apparatus (CerebraMechanics, Lethbridge, Can) according to the published protocol (Wang, Lu et al. 2010, Lu, Morgans et al. 2013). Briefly, rats were placed in the center of the platform, where they can track the grating with reflexive head movements. The spatial frequency of the grating was clamped at the viewing position by re-centering the cylinder on the animal's head. The acuity threshold was quantified by increasing the spatial frequency of the grating until the following response was lost, thereby defining the acuity.

Electroretinography (ERG): Animal was kept in complete darkness overnight to achieve dark-adapted state of the retina. Animal was anesthetized with IP injection of dexmedetomidine/ketamine and placed in a stereotaxic head holder. Under a dim red illumination, the recording electrode (two coaxial wire loops, wire diameter 50 um, attached to the neutral contact lens) was placed on the animal's eye pretreated with Lidocaine. The pupil was dilated with Tropicamide. The eye was stimulated with full-field light flashes (brightness varying from 0.1 to 100 cdm2, duration of 10 ms, interval 3-10 s). Corneal potentials were recorded with the amplifier connected to the electrode. Flash presentations were controlled with a computer program (Spike2 script language, CED Ltd, UK). The responses were averaged for 5-100 stimulus presentations, depending on the ERG strength, which can be very low in animals with progressive retinal degeneration.

### Retinal Histology:

After functional tests at P90, rats were euthanized, and eyes were fixed with 4% paraformaldehyde. Eyes were embedded in OCT and cut into 10 μm sections on a cryostat (Leica CM1950; Leica Microsystems). The sections were collected according to previous protocol (Wang, Lu et al. 2010, Lu, Morgans et al. 2013). One slide from each set was stained with cresyl violet (CV) to assess the injection site and integrity of retinal lamination; the rest of the slides stored at −40° C. were used for antibody staining.

### Immunofluorescent Staining and Confocal Microscopic Imaging

Retinal sections were stained with recoverin (rabbit polyclonal, 1:2,000; Millipore), RPE65 (mouse monoclonal, 1:1,000; Millipore) antibodies using published protocols (Wang, Lu et al. 2010, Lu, Morgans et al. 2013). Anti-mouse or rabbit secondary antibodies conjugated to Alexa Fluor-488 or Alexa Fluor-568 (Life Technologies) were used and counterstained with 49,69-diamidino-2-phenylindole (DAPI). Images were taken with a confocal microscope (Eclipse C1si; Nikon Instruments, Inc., Melville, NY).

### Statistical Analysis:

The results are reported as the mean±SEM (standard error of the mean).

Visual acuity and b-wave amplitudes of NP-CNTF/OSM treated, sham-treated, or untreated eyes were analyzed by one-way ANOVA with Bonferroni post hoc power analyses (GraphPad Prism 5 statistical analysis software). p values <0.05 were considered significant. Asterisks and #signs indicate significance, *, p<0.05; **, p<0.01; ***, p<0.001.

### Results

### 1. Development and Establishment of a Platform for Biological Function Test of CNTF and OSM In Vitro

### The TF-1 Cells are not Suitable to Test the Biological Functions of CNTF and OSM

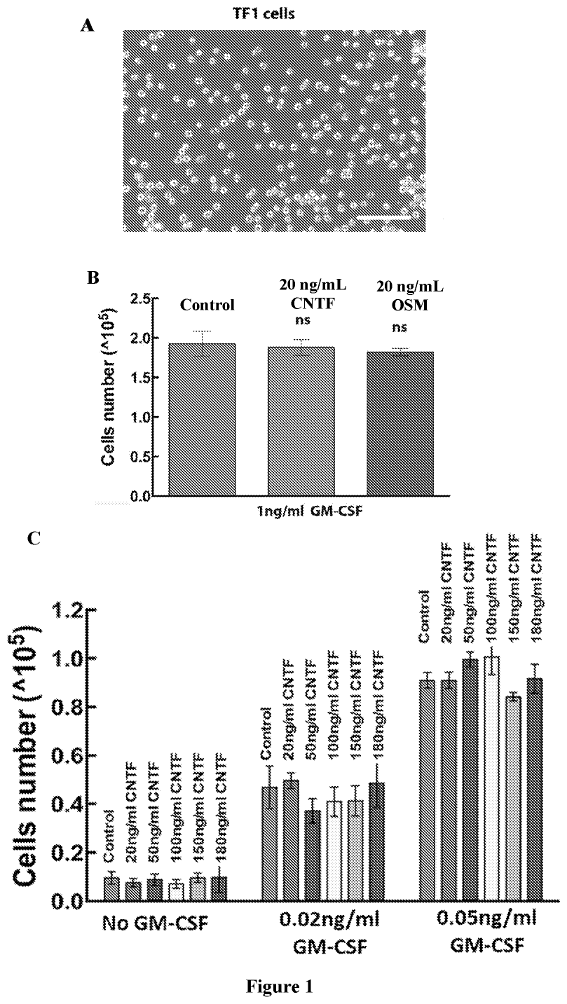

In order to compare the biological functions of native CNTF/OSM and NP-CNTF/OSM in vitro, we started with the TF-1 cell line (FIG. 1, panel A) which has been widely used in proliferation bioassays by manufacturers for their routine quality assurance and quality control of multiple cytokines including CNTF and OSM. For our initial experiment, 8×10³ cells were seeded in suspension in each well of a 24-wells plate in growth media supplemented with GM-CSF (1 ng/ml). Cultures were continuously exposed to 20 ng/ml of native CNTF or OSM for 3-4 days in suspension, then cell numbers were counted. The number of viable cells was used in our study as an indicator of the neurotrophic effect on cell proliferation and cell survival. We found no significant difference in cell numbers between CNTF and OSM treated groups as compared to control group (FIG. 1, panel B), suggesting that 1 ng/ml GM-CSF, which is necessary for long term TF1 cell growth in culture might result in excessive cell proliferation. Therefore, GM-CSF concentration in TF-cell culture was lowered to 0.02 ng/ml and 0.05 ng/ml, and different concentration of native CNTF (20, 50, 100, 150 and 180 ng/ml) were added to the culture media to identify an optimal dose necessary to elicit maximal proliferative effect on TF-1 cells. In the absence of GM-CSF, supplementation of CNTF at any doses up to 180 ng/ml in culture showed no significant proliferation enhancement of TF-1 cells (FIG. 1, panel C). Although supplementation with 0.02 ng/ml and 0.05 ng/ml GM-CSF lead to an approximately 5- and 10-fold increase of cell numbers, respectively; addition of CNTF or OSM (data not shown) in the culture media did not have any effect on cell proliferation at all within a wide range of doses (FIG. 1, panel C). These results demonstrated that TF-1 cells, although have been used in previous studies, are not suitable for testing the biological effect of, at least, CNTF and OSM in vitro.

Generation of Human iPSC-Derived Eye Field Progenitors(EFP), Retinal Neural Progenitors(RNP)

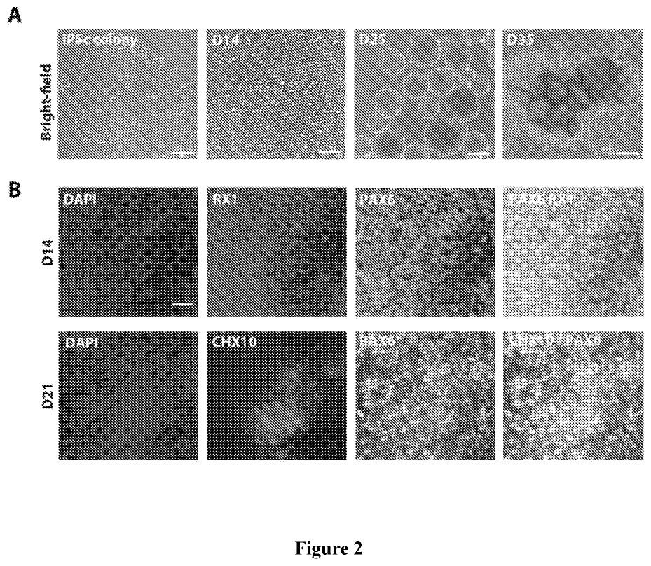

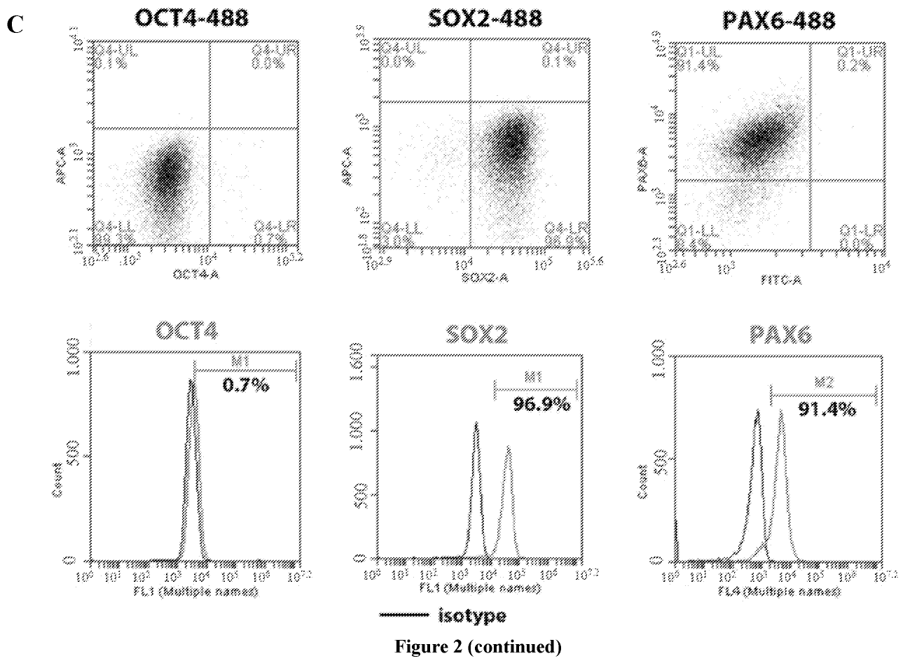

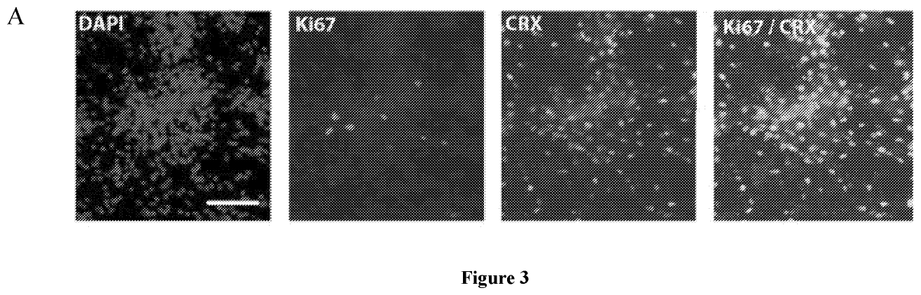

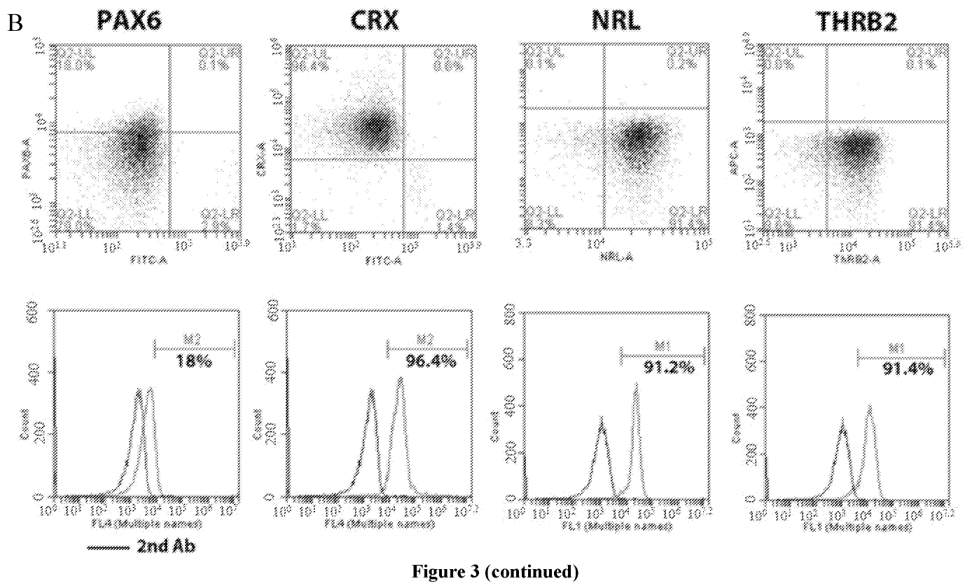

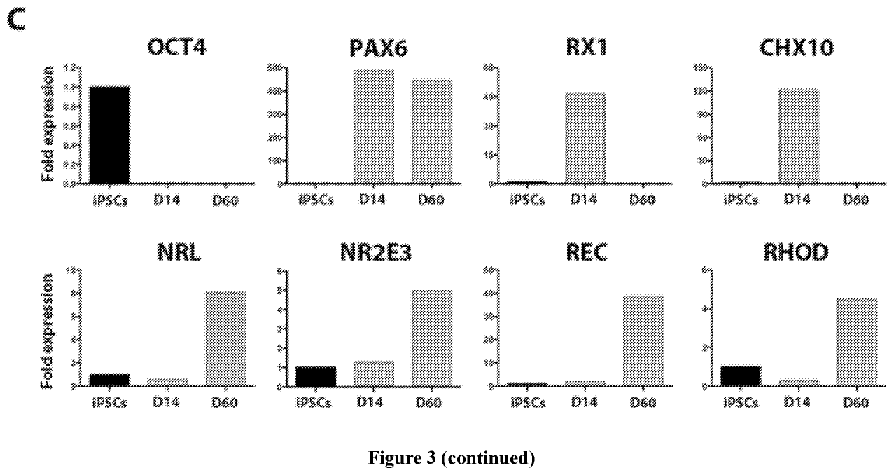

In order to obtain a cell population that will be suitable for testing the biological functions of CNTF and OSM, we developed a protocol to generate highly pure retinal photoreceptor progenitors (RPPs) from human iPS cells. Stepwise lineage specific differentiation of iPS cells were induced in vitro toward RPP population. The appearance of eye field progenitors (EFPs) within the primitive neuroepithelium was the first phase in a step-wise production of retinal cells from undifferentiated iPSCs (FIG. 2, panel A). In the current study, EFPs were differentiated as an adherent monolayer, followed by retinal neural progenitors (RNPs) as neurospheres in suspension formed by mechanical detachment. Neurospheres were subsequently attached on Laminin-511 coated surfaces to promote neural rosettes formation, resembling eye cup structures (FIG. 2, panel A). During this process, rapid upregulated (Day 14) expression of pan neural induction marker Paired box 6 gene (PAX6; 91.4% by flow cytometry) and a transcription factor associated with EFP specification, retinal homeobox gene 1 (RX1) were observed (FIG. 2, panel B and panel C); while the expression of the pluripotent octamer-binding transcription factor 4 gene (OCT4; 0%) was completely shut down (FIG. 3, panel C). The high expression of Sex determining region Y-box 2 (SOX2; 96.9%), a general neural marker, was also an indication of successful neural induction (FIG. 2, panel C). Ceh-10 homeodomain containing homolog (CHX10), a marker shown to be important in the proliferation and differentiation process during retinogenesis in vivo, was also detected at day 21 by immunohistochemistry in a large percentage of PAX6 positive cells, indicating the acquisition of RNP phenotype (FIG. 2, panel B).

Efficient Generation of Human iPSC-Retinal Photoreceptor Precursors (iPS-RPP)

Differentiation toward early retinal Photoreceptor progenitors(RPPs) was supported by the emergence of the Photoreceptor progenitor-specific transcription factor Cone-Rod homeobox (CRX) at Day 60 (FIG. 3, panel A). Immunohistochemistry showed that nearly all DAPI positive cells within our cultures were positive for CRX, while Ki67 (a general proliferative marker), was nearly absent, suggesting the acquisition of a post-mitotic cell identity of RPPs (FIG. 3, panel A). Further confirmation of photoreceptor identity was illustrated by flow cytometry analyses at D60 (FIG. 3, panel B), whereby downregulation of the general neural markers PAX6 (18%) was associated with a significant increase in the expression of markers important for the transcriptional regulatory cascade in human retinal development, such as CRX (96.4%), neural retina leucine zipper (NRL) (91.2%), and thyroid hormone receptor β2(TrRB2) (91.4%). To characterize in more detail the molecular identity of retinal cells induced step-wise from human iPSCs, real-time quantitative reverse transcription polymerase chain reaction (RT-qPCR) analyses (FIG. 3, panel C) further revealed the undetectable levels of OCT4 expression at D14, together with an early upregulation of PAX6 and RX1 expression at D8 (data not shown), which continued at D14. CHX10 expression was also upregulated at D14, indicative of RNPs lineage. While PAX6 expression declined slightly at D60, the expression of RX1 and CHX10 was completely shut down, suggesting a progression from EFPs and RNPs stages to a more differentiated stage toward photoreceptors development. As expected, the expression of rod specific RPPs markers NRL, nuclear receptor subfamily 2, group E, member 3 (NR2E3), recoverin and rhodopsin was specifically elevated at D60 as compared to D14. Taken together, these analyses confirmed the highly efficient and nearly homogeneous generation of RPPs from human iPSCs.

Both CNTF and OSM are Pro-Survival and Pro-Proliferation Factors for iPS-RPPs

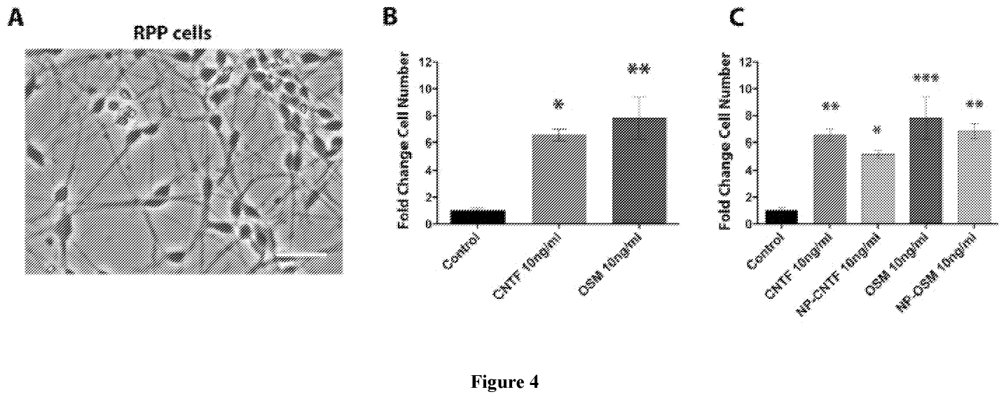

We next tested whether human iPS-RPPs are responsive to CNTF and OSM treatment, which may serve as a platform to evaluate their biological functions in vitro. Single cells were dissociated from neurospheres at D60 of differentiation and seeded in NDM media onto Matrigel coated 24-wells plates at a density of 5×10⁴ cells/well. Morphological examination of cells prior to initiation of the treatment shows a large network of processes emanating from neuronal cells bodies (FIG. 4, panel A). Following a 48 hours period of attachment and recovery, cell cultures were treated with 10 ng/ml of CNTF and OSM in triplicate. No treatment control group was used in triplicates for comparison purposes. Half medium was changed every 3-4 days while maintaining a constant CNTF and OSM concentration. After D21 in culture, cells were counted as an indicator of proliferation. We found a significant increase (>5-fold) in cell numbers for both CNTF and OSM treatments (CNTF group, 6.56±0.43×10⁵ and OSM group, 7.82±1.6×10⁵) as compared to no treatment normalized control (1±0.16×10⁵) (FIG. 4, panel B). In contrast, cell numbers decreased in no treatment control group. These results demonstrate that iPS-RPP cells are a better cell population than the TF-1 cells for assessing the biological functions of CNTF and OSM in vitro.

NP-CNTF and NP-OSM Possess Identical Biological Functions on iPSC-RPPs as their Native Counterparts In Vitro

We next wanted to test whether, after being incorporated into biocompatible nanoparticles, CNTF and OSM (NP-CNTF and NP-OSM) retain their biological functions, at least, on human iPS-RPPs. As described above, cell cultures were treated with 10 ng/ml of CNTF and OSM, as well as 10 ng/ml of NP-CNTF and NP-OSM in triplicate side by side (FIG. 4, panel C). After D21 in culture, cells were counted as an indicator of proliferation. We found no significant difference between NP-proteins and their native proteins (CNTF, 6.56±0.43×105 vs. NP-CNTF, 5.19±0.25×105; OSM, 7.82±1.47×105 vs OSM-NP, 6.84±0.52×105), confirming that both native growth factors and their modified proteins have comparable biological potency.

NP-CNTF and NP-OSM Promote the Survival and Proliferation of iPS-RPP Cells Derived from Different Human iPSC Lines

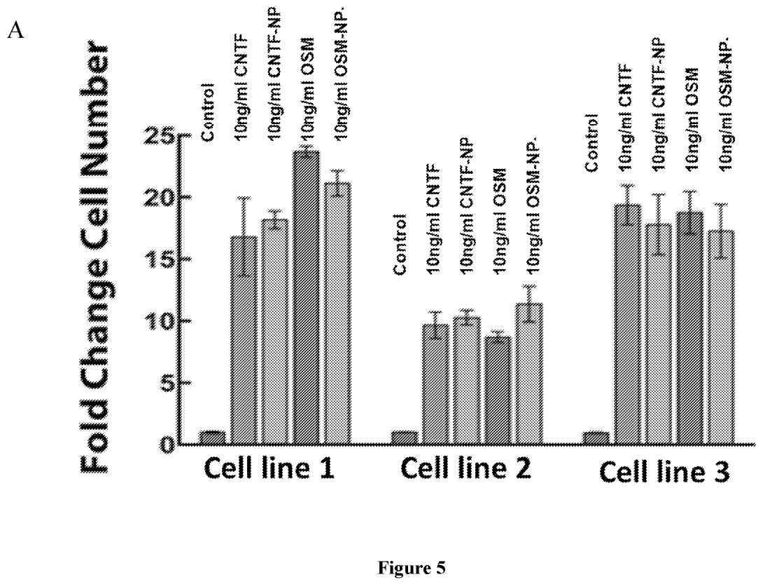

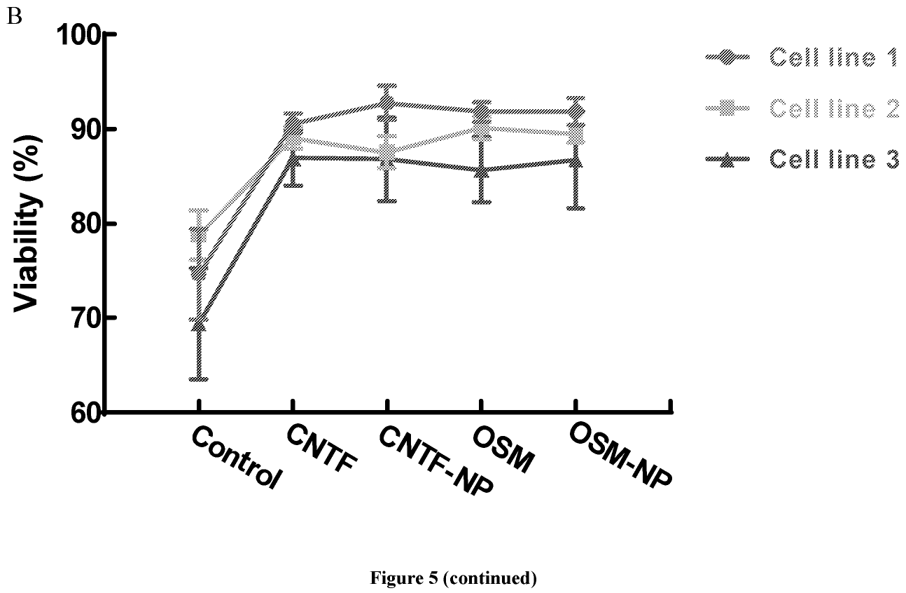

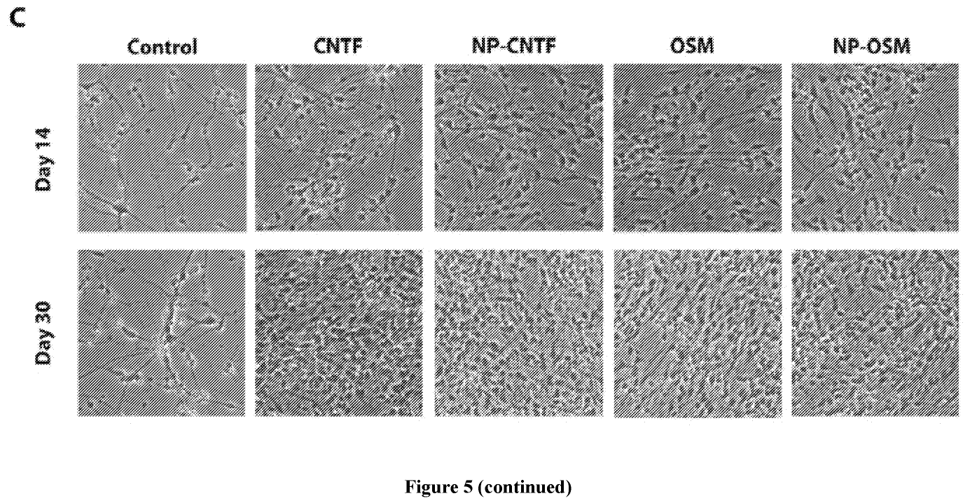

To validate whether the observed pro-survival and pro-proliferation effects of NP-CNTF and NP-OSM are reproducible, 3 different iPS-RPP preparations derived from 3 different iPSC lines were examined (FIG. 5). iPS-RPPs from 3 different iPSC lines were exposed to 10 ng/ml of CNTF, NP-CNTF, OSM, and NP-OSM in triplicates, cell numbers and viabilities were examined as described above. Compared to controls, treatments with CNTF, NP-CNTF, OSM, and NP-OSM lead to significant increases in normalized cell numbers (FIG. 5, panel A), as well as cell viabilities (FIG. 5, panel B) for all three cell lines tested. No difference was observed between native proteins and NP-proteins, consistent with our earlier observations. To determine whether morphological changes occur during the entire course of the treatment, and to asses for the optimal time for performing the quantitative analysis, we monitored iPS-RPP cultures by acquiring bright-filed images every 7 days (FIG. 5, panel C). As expected based on cell counting and viability results, at day 30 after treatment initiation, the control untreated cultures displayed a rather sparse population with limited cell-cell connections and processes, while all the treated cultures show a densely packed network of neural connections associated with an abundance of cell bodies with “healthy” appearance, due to the neuroprotective effects of neurotrophic factors. Based on our qualitative investigation of morphological changes, we determined that the onset of changes started to occur at Day 14 post treatment, with a gradual increase in the survival and proliferating effect of growth factors by Day 30 of treatment (FIG. 5, panel C). These results validate our human iPS-RPP platform for assessing the biological functions of neurotrophic factors in vitro.

Efficient Generation and Characterization of hiPSC-Retinal Ganglion Progenitors (iPS-RGPs) and Biological Effect of CNTF, NP-CNTF, OSM, and NP-OSM on iPS-RGPs

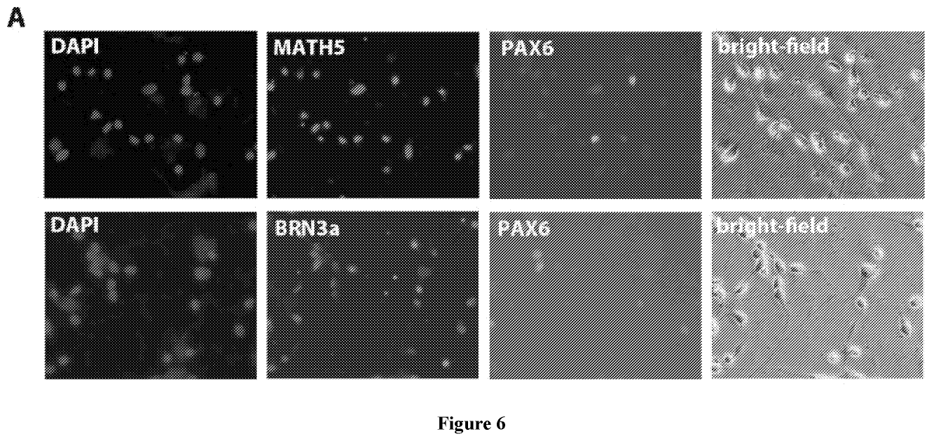

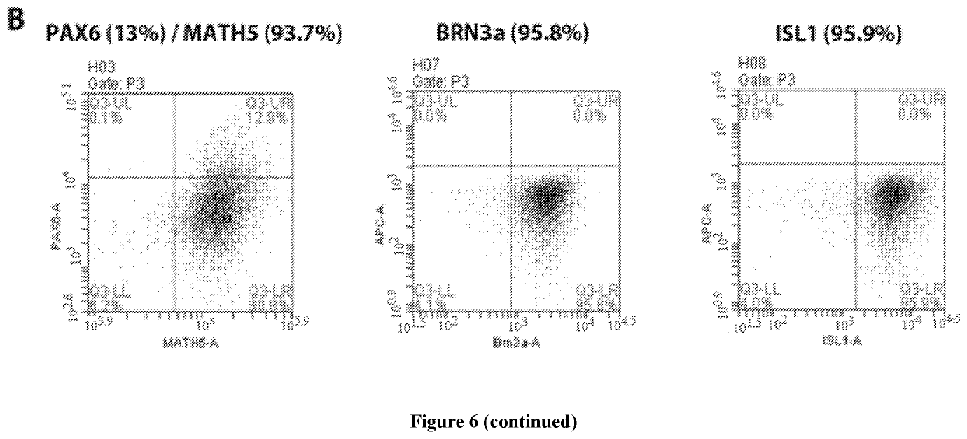

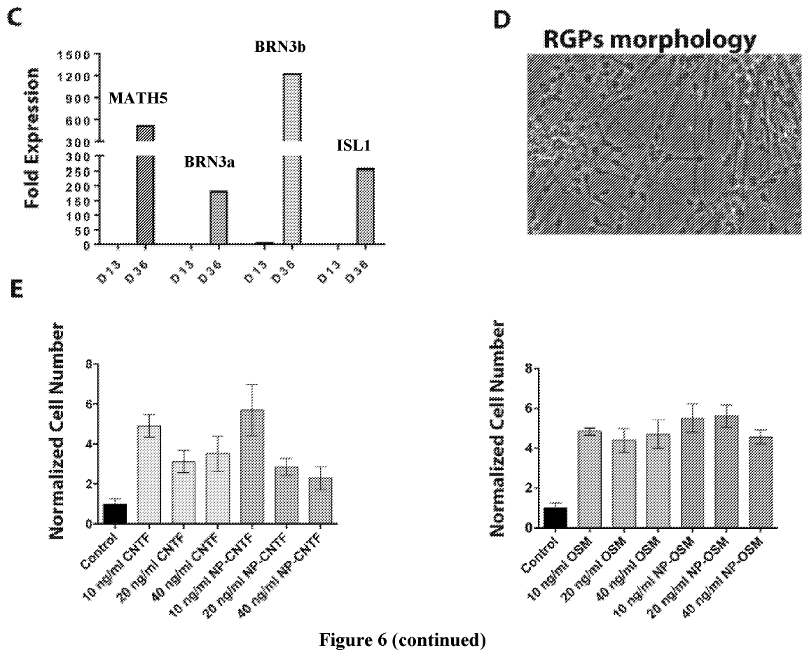

As described in Methods and shown in FIG. 2, by D14 of differentiation, the eye field cell transcription factors, PAX6 and RX1, were highly expressed in most cells. At day 35, immunostaining of cells clearly revealed downregulation of PAX6 (green) and increased expression of RGPs markers, MATH5 (bHLH transcription factor) and Bm3a (a member of the POU-domain family transcription factor), which have been shown to be crucial for RGP development (FIG. 6, panel A). Flow cytometry analysis further reveled the successful generation of a highly pure population of RGPs, as illustrated by the low expression of PAX6 (13%), and high percentage of staining with MATH5 (93.7%) and BRN3a, (95.8%). ISL1 (Islet1), a LIM-homeodomain factor, which is another reliable marker for ganglion progenitor cells was also upregulated in RGPs (95.9%) (FIG. 6, panel B). Immunostaining and flow cytometry results were consistent with the temporal expression of all four markers in RGPs as determined by quantitative qPCR analysis, which revealed low gene expression at D13 prior to gangliogenesis, and then dramatically upregulated at D35 in early RGPs (MATH5, BRN3a, BRN3b, and ISL1) (FIG. 6, panel C). We therefore examined the biological functions of CNTF, NP-CNTF, OSM and NP-OSM (at 10, 20, and 40 ng/ml each in triplicates) on iPS-RGPs, which showed an elaborated network of neuronal bodies and axons (FIG. 6, panel D). We observed a significant increase in cell numbers for both native CNTF and OSM as well as NP-CNTF and NP-OSM at all concentrations tested (FIG. 6, panel E), although a decrease in cell number at high doses (20 and 40 ng/ml) of CNTF and NP-CNTF as compared to lower dose (10 ng/ml). Our results demonstrate that NP-CNTF and NP-OSM promote the survival and proliferation of iPS-RGP, indicating the potential prevention of optical nerve death and glaucoma for patients.

### 2. Vison Rescue in a Rat Model of Photoreceptor Degeneration by Intravitreal Delivery of NP-CNTF and NP-OSM

Intravitreal delivery of NP-CNTF and NP-OSM preserved visual function in RCS rats

In order to determine whether NP-CNTF and NP-OSM can rescue visual functions in animals with inherited retinal degeneration, we used the Royal College of Surgeons (RCS) rat as a model. In this animal, the retinal pigment epithelial cell (RPE) fails to phagocytose shed outer segment material at a normal rate because of a mutation in the c-mer protooncogene tyrosine kinase (Mertk) gene. This results in an accumulation of undigested toxic outer segment debris in the subretinal space between the outer nuclear layer (ONL) and RPE and subsequently leads to progressive photoreceptor death and commensurate visual loss.

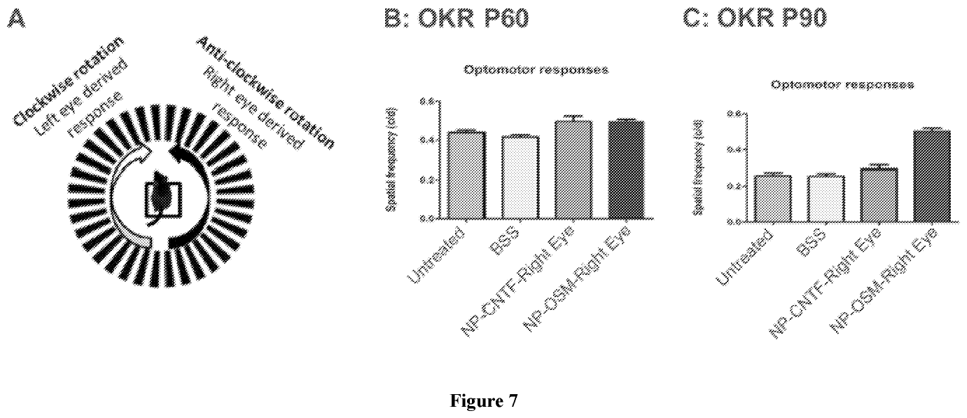

Visual functions were assessed in eyes that received NP-CNTF, NP-OSM, and control NP only or left untreated at P60 and P90. Spatial visual acuity in units of cycle/degree (c/d) assessed by optokinetic response (OKR) was significantly higher in both NP-CNTF and NP-OSM-treated eyes compared with control NP treated and untreated control eyes at P60 (FIG. 7, panel B). Visual acuity decreased substantially between P60 and P90 in untreated, control NP treated (p<0.001) and NP-CNTF treated eyes, while OKRs showed a sustained visual acuity in NP-OSM-injected eyes between the same time points (FIG. 7, panel C), a substantial delay of visual function decline is evidenced in NP-OSM treated eyes.

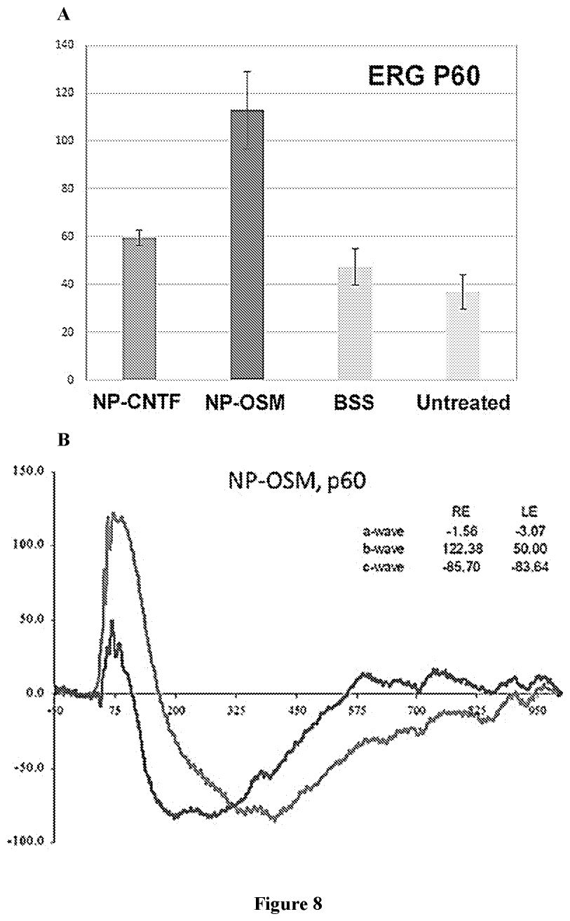

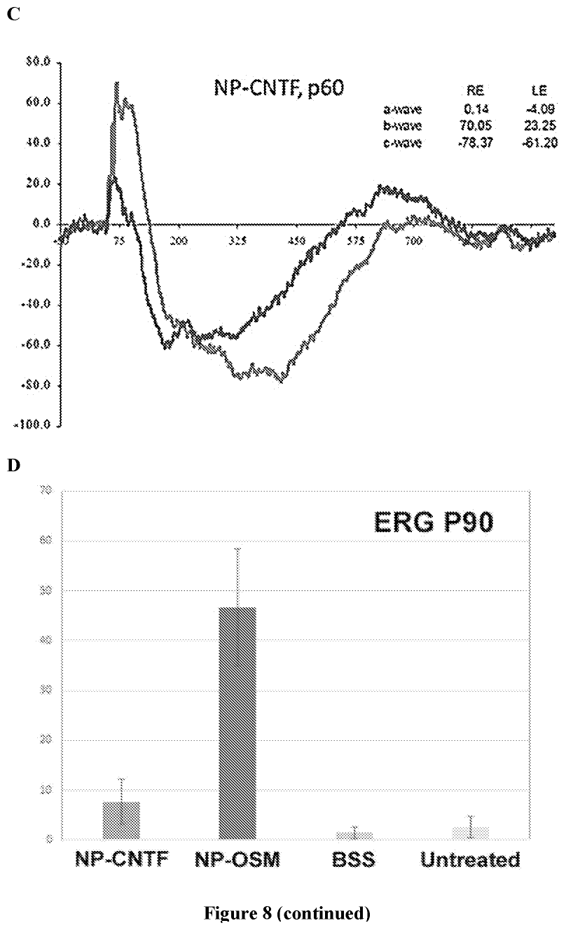

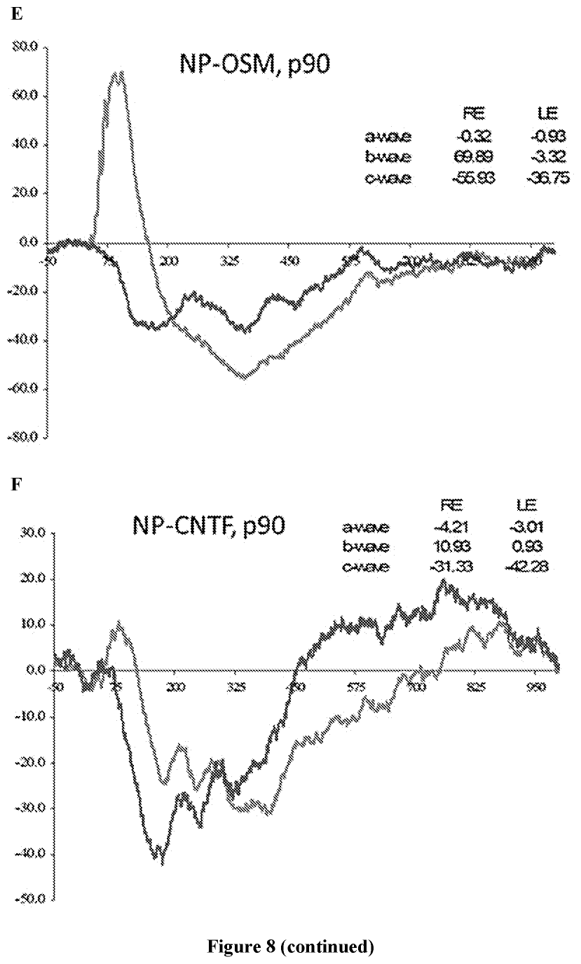

In addition, retinal electrical responses to light stimulation were measured by electroretinography (ERG). As shown in FIG. 8, panels A-C, b-wave amplitudes following NP-CNTF and NP-OSM administration were significantly higher when compared with those of sham treated and untreated control eyes at P60, and NP-OSM treated eyes showed a much better ERG preservation than NP-CNTF treated eyes. ERG b-wave amplitudes in general decreased over the time between P60 and P90 in all eyes, but NP-OSM-injected eyes showed a substantial ERG preservation compared to NP-CNTF, control NP and untreated eyes, suggesting that NP-OSM possess the ability to halt or slow down retinal degeneration long term.

In conclusion, functional test data for OKR and ERG at both P60 and P90 have shown a clear difference between NP-OSM-treated and contralateral untreated eyes, confirming a single intravitreal administration of nanoparticle incorporated NP-OSM has a long lasting effect to preserve the vision loss caused by degeneration of photoreceptor layer and perhaps the underlining RPE layer as well.

Intravitreal injection of NP-OSM preserved the integrity of whole retina

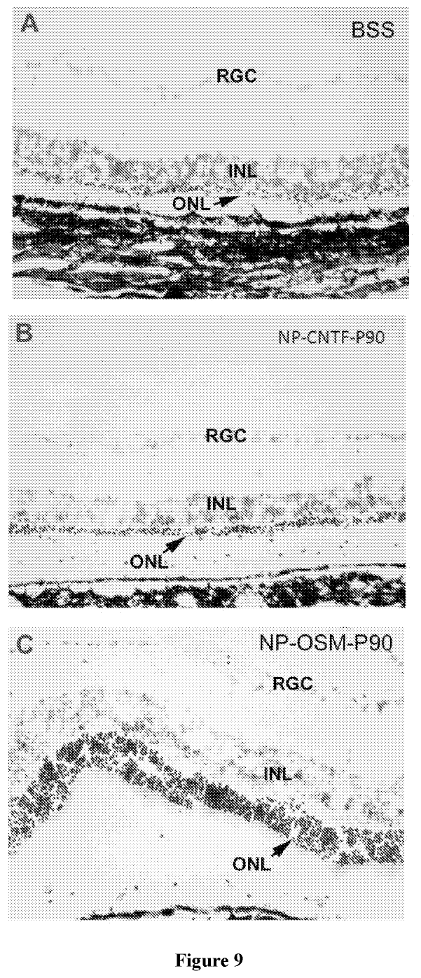

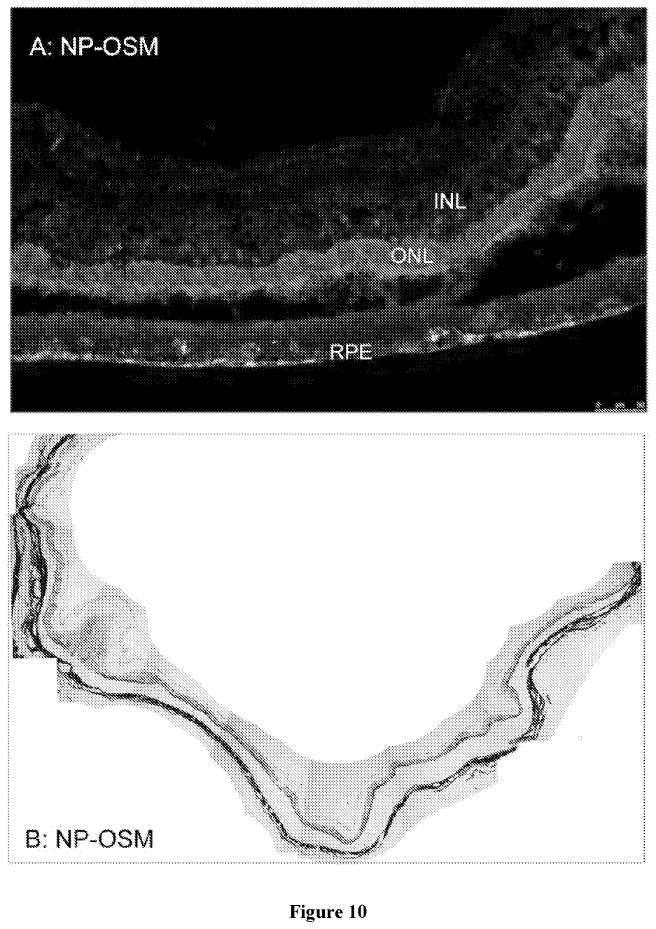

To confirm whether NP-CNTF and NP-OSM preserved photoreceptors, frozen retinal transverse sections were stained with cresyl violet from eyes that received NP-CNTF, NP-OSM injection or eyes left untreated. A remarkable protection of photoreceptors in NP-OSM treated eyes was observed at P90 with an ONL thickness of 8-10 nuclei layers (FIG. 9, panel C). This was in contrast to the ONL with just one discontinuous row of photoreceptors in untreated eyes (FIG. 9, panel A) and thickness of 2-3 nuclei layers in NP-CNTF treated eyes (FIG. 9B). The preservation of NP-OSM is global: one intravitreal injection protected the whole retina from degeneration. (FIG. 10, panel B). The well preserved ONL layer was characterized by immunostaining for recoverin (Red) in NP-OSM-treated retinas (FIG. 10, panel A). More importantly, an intact RPE (anti-RPE65, green, FIG. 10, panel B) layer was observed in NP-OSM treated retina, which is in contrast to hypertrophic and disorganized RPE layer observed in control P90 RCS retina as demonstrated in earlier studies (Tsai, Lu et al. 2015), suggesting that the protective effect of NP-OSM is not limited only to retinal neurons, but extended to RPE layer as well, preserving the integrity of the whole retina.

In summary, these in vivo results demonstrate that a single intravitreal injection of biocompatible NP-OSM is able to preserve the visual functions in inherited photoreceptor degeneration RCS rats long term. Histological analysis confirms that NP-OSM treatment prevented retinal photoreceptor degeneration, and importantly RPE layer of NP-OSM treated retina displayed no sign of structural degeneration at P90, which is in contrast to earlier observations that RPE layer damage and hypertrophy are typically present in RCS rats at the comparable stage. (Tsai, Lu et al. 2015)

### Discussion

In this study, we have developed and established a platform that can be used to test the biological functions of neurotrophic factors such as CNTF and OSM in vitro, and this human iPS-RPPs and iPS-RGPs based platform may be able to extend to other factors and their derivatives. Using this novel platform, we demonstrated that the biocompatible NP-CNTF and NP-OSM possess identical biological functions as their native counterparts in promoting human iPS-RPP and iPS-RGP viability and proliferation in vitro. Furthermore, we also demonstrated for the first time that stable and biocompatible nanoparticle conjugated CNTF and OSM (NP-CNTF and NP-OSM), when administered into the vitreous of inherited retinal degeneration RCS rats, preserved the visual function and retinal integrity of these animals, which represent a model of several human retinal diseases such as retinitis pigmentosa (RP), Stargardt disease and age-related macular degeneration (AMD) that are the leading cause of human blindness. As both NP-CNTF and NP-OSM, especially the latter, showed neuroprotective effects in vivo, these observations may extend to other retinal neurodegenerative diseases such as glaucoma and general neurodegenerative diseases such as Alzheimer's and Parkinson's diseases.

Photoreceptor degeneration is responsible for the onset of retinitis pigmentosa (RP). Stargardt disease and age-related macular degeneration (AMD) are initially manifested by the degeneration of retinal pigmented epithelial (RPE) cells which provide functional support to photoreceptors. The progression of the disease eventually leads to the loss of photoreceptors. Degeneration of photoreceptor and hypertrophy of RPE are well documented in geographic atrophy (GA), an advanced stage AMD (Sunness, Gonzalez-Baron et al. 1999). Preserving the health of photoreceptors as well as RPE is the key to stop disease progression and maintenance of functional vision in RP, Stargardt disease and AMD patients. Drugs selected based on their protective effects on RPPs will not only benefit RP, Stargardt disease and AMD patients, but diabetic retinopathy patients as well. As shown in our in vitro study, similar protecting effects on iPS-RGP was obtained, indicating the potential for the treatment of glaucoma.

In vitro derivation of specific cell lineages from pluripotent stem cells offer a powerful platform for drug screening. Primary culture of human retinal Photoreceptor progenitors is extremely difficult to establish due to limited access to material, lack of suitable culture condition to sustain cell growth and phenotype in vitro. Using our newly developed process for derivation of RPP cells from human iPS cells, we can reproducibly generate 50 iPS-RPP cells from every starting iPS cells under defined culture condition. The iPS-RPP cells can be easily cryopreserved in large quantity and displayed high recovery rate post thaw (data not shown). It is therefore suitable for the development of large scale high throughput drug screen platform. Our results strongly suggest that a well-established in vitro drug discovery platform using iPSC-derived cell lineage will facilitate the emergence of a new generation of precision medicine and reduce the overall cost of future drug development.

Despite the promise of emerging cell replacement therapies, treating retinal diseases with neurotrophic factors has increasingly been an attractive therapeutic pathway. The major challenge is the lack of suitable technology allowing long term sustained release of optimal dose of growth factors without potential negative impact on the microenvironment surrounding the retina. Genetically modified human fibroblasts overexpressing CNTF were encapsulated and used as an intraocular implant for 24 months. Although data from phase 1 clinical trials showed visual acuity improvement, subsequent phase 2 studies failed to demonstrate any short-term or long-term improvement. In fact, the intravitreal implant is implied to be responsible for loss of total visual field sensitivity in treated eyes (Birch, Bennett et al. 2016). The clinical outcome of these early studies highlighted the importance of a suitable and biocompatible long-term growth factor delivery method into a such sensitive organ. Implants carrying cells secret many factors other than CNTF. It is therefore very hard to draw meaningful conclusion on the overall negative outcome of the phase II investigation. We hypothesis that intravitreal injection of single neurotrophic factor in a new form of biocompatible long-lasting nanoparticle formulation will provide much improved outcome.

Here, in our in vitro assay, both NP-CNTF and NP-OSM were well tolerated by iPS-RPPs and iPS-RGPs for up to 4 weeks in culture, indicating no cytotoxity. It is 100% water soluble and transparent which is ideal for use in the delicate intravitreal microenvironment to ensure long lasting effect of the neurotrophic factors. The intravitreal administration of NP-CNTF and NF-OSM in RCS rats were well tolerated, and visual function rescue and retinal integrity preservation were also observed in these animals. The success of locally delivered long-lasting nanoparticle incorporated neurotrophic factors will not only benefit patients suffering retinal diseases, it can be applied to other neurodegenerative diseases such as Parkinson's and Alzheimer's disease.

Unlike CNTF, the clinical efficacy of OSM on retinal diseases such as RP and AMD have not been investigated. OSM is a pleiotropic cytokine with much diverse functions than CNTF. OSM is generally believed to be important in liver development, hematopoiesis, inflammation and central nerves system (CNS) development. It is also associated with bone formation and destruction (Walker, McGregor et al. 2010), and plays a role in cardiomyocyte proliferation (Kubin, Poling et al. 2011). Using the S334ter-3 rat model of retinal degeneration, Xia et al reported that recombinant OSM protected both rod and cone photoreceptors. In addition, OSM regenerated cone outer segments in early stages of cone degeneration (Xia, Li et al. 2011). Similarly the same group also showed that intravitreal injection of free OSM protected pattern ERG and RGC in a optic nerve injury mouse model (Xia, Wen et al. 2014). Our study is the first to demonstrate direct pro-survival and pro-proliferation effect of OSM on human retinal neural progenitor cells in vitro. Furthermore, vitreous delivery of NP-OSM also demonstrated long-term visual preservation in RCS rats, overcame the hurdle of frequent intravitreal injection. Although both CNTF and OSM are member of the IL-6 cytokine family, there are significant difference in their receptor binding specificity as well as biological function (Rose-John 2018). In S334ter-3 rat model, the protective effect of OSM is indicated to be mediated by Muller cells rather than act directly on photoreceptor cells (Xia, Li et al. 2011). Although it is unclear how OSM stimulate growth of culture human iPS-RPPs and iPS-RGPs in vitro, it is possible to directly bind to its receptor and initiate the signal transduction.

We also speculate that the mechanism behind retinal protective effect of OSM could be different from that of CNTF in the same system. Using OSM instead of CNTF may overcome some of shortcomings of unsuccessful clinical investigation in treating retinal degeneration.

The development of therapeutic drugs for neurodegenerative diseases has been disappointed so far, for which no treatment showed any efficacy of reversing the symptoms for these patients. From developmental biology point of view, prevention, such as halt or slow down the progression of these diseases, may be more practical. Consistent delivery of neuroprotective agents such as NP-OSM through vitreous injection may be a practical choice to control retinal neurodegeneration, and the same approach may extend to other neurodegenerative diseases, such as Alzheimer's disease and Parkinson disease, by precisely controlled delivery of NP-OSM.

### Example 2: Investigation of therapeutic effect in vivo of nanoparticle incorporated OSM on Alzheimer's Disease animal model

We have demonstrated that stable and biocompatible NP-OSM, and NP-CNTF in a less degree, are neuroprotective both in vitro and in a genetic retinal degeneration animal model, indicating a strong therapeutic value for retinal neurodegeneration diseases. In this Example, we focus to investigate neuroprotective effect of NP-OSM and NP-CNTF on neurodegenerative diseases of central nerve system, Alzheimer's disease (AD).

AD is characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus (Wenk, G. L. 2003. ‘Neuropathologic changes in Alzheimer's disease’, J Clin Psychiatry, 64 Suppl 9: 7-10).

Alzheimer's disease has been identified as a protein misfolding disease (proteopathy), caused by plaque accumulation of abnormally folded amyloid beta protein, and tau protein in the brain (Hashimoto, M., E. Rockenstein, L. Crews, and E. Masliah. 2003. ‘Role of protein aggregation in mitochondrial dysfunction and neurodegeneration in Alzheimer's and Parkinson's diseases’, Neuromolecular Med, 4: 21-36). Plaques are made up of small peptides, 39-43 amino acids in length, called amyloid beta (Aβ). Aβ is a fragment from the larger amyloid precursor protein (APP). APP is critical to neuron growth, survival, and post-injury repair (Priller, C., T. Bauer, G. Mitteregger, B. Krebs, H. A. Kretzschmar, and J. Herms. 2006. ‘Synapse formation and function is modulated by the amyloid precursor protein’, J Neurosci, 26: 7212-21; Turner, P. R., K. O'Connor, W. P. Tate, and W. C. Abraham. 2003. ‘Roles of amyloid precursor protein and its fragments in regulating neural activity, plasticity and memory’, Prog Neurobiol, 70: 1-32). Alterations in the distribution of different neurotrophic factors and in the expression of their receptors such as the brain-derived neurotrophic factor (BDNF) have been described in AD (Schindowski, K., K. Belarbi, and L. Buee. 2008. ‘Neurotrophic factors in Alzheimer's disease: role of axonal transport’, Genes Brain Behav, 7 Suppl 1: 43-56; Tapia-Arancibia, L., E. Aliaga, M. Silhol, and S. Arancibia. 2008. ‘New insights into brain BDNF function in normal aging and Alzheimer disease’, Brain Res Rev, 59: 201-20).

Animal model for AD

B6.Cg-Tg(APPswe,PSEN1dE9)85Dbo/Mmjax (APP/PS1): Double transgenic mice express a chimeric mouse/human amyloid precursor protein (Mo/HuAPP6(95swe) and a mutant human presenilin 1 (PS1-dE9) both directed to CNS neurons. Both mutations are associated with early-onset Alzheimer's disease (Jankowsky, J. L., D. J. Fadale, J. Anderson, G. M. Xu, V. Gonzales, N. A. Jenkins, N. G. Copeland, M. K. Lee, L. H. Younkin, S. L. Wagner, S. G. Younkin, and D. R. Borchelt. 2004. ‘Mutant presenilins specifically elevate the levels of the 42 residue beta-amyloid peptide in vivo: evidence for augmentation of a 42-specific gamma secretase’, Hum Mol Genet, 13: 159-70).

Transgenic mice develop beta-amyloid deposits in the brains of transgenic animals by 6 to 7 months of age.

### Experimental Plan

APP/PS1 at 3 months in age were injected intracranially with or without NP-OSM/NP-CNTF as grouped as following:

| | | |
|---|---|---|
| 1) | APP/PS1 mice (x5): | No injection control |
| 2) | APP/PS1 mice (x5): | NP particle sham injection |
| 3) | APP/PS1 mice (x5): | NP-OSM (10-30 mg/mouse) |
| 4) | APP/PS1 mice (x5): | NP-CNTF (10-30 mg/mouse) |

The progressive development of AD and potential therapeutic effect of NP-OSM/NP-CNTF will be evaluated as following experimental procedures:

Behavior Study: Use video-EEG to monitor seizure and epileptiform discharges from 3 to 17-18 weeks in age, as detected by video-EEG.
Mortality: Mortality rate of all animal group will be monitored.
Histology: Brains of all animals involved in the study will be collected and subjected to whole mount section and subsequent histological analysis of beta-amyloid deposits and plaque accumulation. Control wild-type mice (C57 Black) at the same age will also be analyzed.

### Example 3: Investigation of NP-trophic factors for protection of retinal ganglion cells in an acute optic nerve crush rat model

Optic neuropathy is a generic term for optic nerve diseases, which lead to irreversible vision loss due to optic nerve damage and death of retinal ganglion cells (RGCs). The most common form of optic neuropathy is glaucoma, affecting 70 million people worldwide. Glaucoma is a group of optic neuropathies with clinical manifestations including cupping of the optic disc, thinning and loss of the retinal nerve fiber layer, and characteristic visual field defects (Weinreb et al., 2014). It is the leading cause of irreversible blindness worldwide (Davis et al., 2016). The initial site of injury in glaucoma is believed to be at the optic nerve head. Glaucomatous changes to the optic nerve head damage the axons of RGC, which are the output neurons of the retina that carry visual signals to the retino-recipient regions of the brain. There is no treatment available to halt glaucoma progression or reverse the damage done to the RGCs. Therefore, early disease detection and treatment aimed at neuroprotection and regeneration are an imminent need

The optic nerve crush (ONC) injury model is an important experimental disease model for traumatic optic neuropathy, glaucoma, etc. and is a common method used to study the effect of axon injury (Templeton et al., 2012; Tang et al., 2011). In this model, the crush injury to the optic nerve leads to gradual RGC apoptosis, which can be used to study the general processes and mechanisms of neuronal death and survival. In addition, pharmacological and molecular approaches can be used in this model to identify and test potential therapeutic reagents to treat different types of optic neuropath. In this proof of concept study, we investigated the neuroprotective effect of nanoparticle-incorporated trophic factors ciliary neurotrophic factor (NP-CNTF) and Oncostatin M (NP-OSM) by intravitreal delivery immediately after ONC procedure in rats, aiming for prevention of RGC apoptosis and death.

### Optic Nerve Crush (ONC) Rat Model

Long Evans rats, both male and female at 8-10 weeks were used for this study. The animals were anesthetized via IP injection of anesthetic agents, one eye is operated, the fellow eye was used as un-operated. The right optic nerve (ON) was surgically exposed in its intraorbital segment, which spans 2 to 3 mm beyond the eye cup, taking care to leave the retinal vascularization intact.

The superior extraocular muscles were spread to allow access to the ON. An incision was made in the eye-retractor muscle and the meninges perpendicular to the axonal orientation, extending over one third of the dorsal ON. The ON was pinched with a customized forceps at a distance of 2 mm behind the eye, leaving the retinal vascularization unaffected. All microsurgery was performed with the aid of a surgical microscope. The ON head was examined ophthalmoscopically immediately after surgery, to ensure that the retinal vasculature was intact. Animals were given the followings via intravitreal injection (4 μl) immediately after ON crush: NP-CNTF (n=8), NP-OSM (n=9) and nanoparticle (NP) control (n=6). Two weeks after ONC, animals were euthanized, retinal whole mount was prepared and stained with retinal ganglion cell (RGC) marker-Brn3 and RGC axons-RT97. Images were taken under fluorescence microscope. Twelve images/retina were taken to count RGCs. The statistical analysis was performed with ANOVA, p<0.05 was considered as significance.

### Results

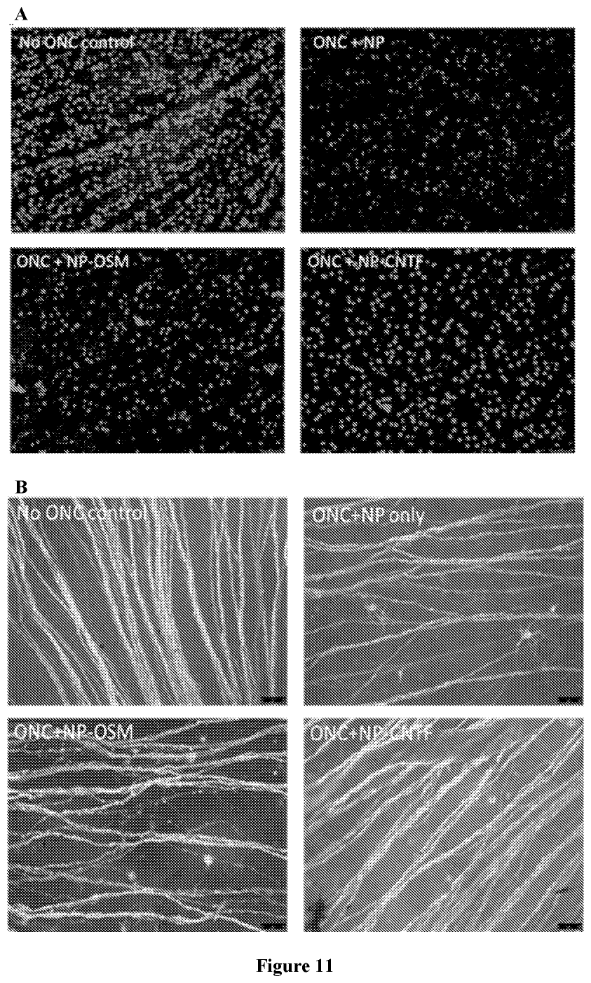

Two weeks after treatments in the ONC with different treatments, retinal whole flat mounts were stained with RGC specific marker Brn3 and analyzed using fluorescent microscope. These images clearly showed a RGC protection by both NP-CNTF and NP-OSM treatments as compared to NP only administration (FIG. 11, panel A), whereas the numbers of RGCs in eyes of native control are more than theses in all ONC eyes treated with different nanoparticles, indicating the protection is partial. Staining with RGC axon specific marker RT97 showed similar results (FIG. 11, panel B).

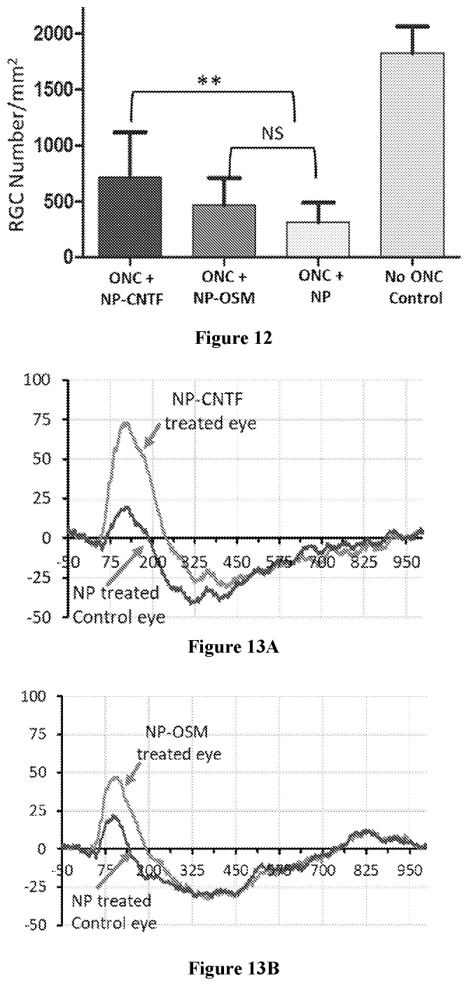

The numbers of RGCs in ONC mounts treated with different nanoparticles were then counted. As shown in FIG. 12, NP-CNTF treated eyes showed significant RGC survival compared with NP-treated eyes (p<0.05), while the difference between NP-OSM and NP-treated eyes did not reach statistically significance, however, substantial protection was observed (FIG. 11). We also observed that RGCs in the intact fellow eyes were significantly higher than any of the treated eyes, suggesting the protection is partially effective in this acute ONC model, consistent with the observations with immunostaining of RGC specific marker Brn3 and RGC axon specific marker RT97 shown in FIG. 11.

### Discussion

Injury to the optic nerve can lead to axonal degeneration, followed by a gradual death of RGCs, which results in irreversible vision loss. Examples of such diseases in human include traumatic optic neuropathy and optic nerve degeneration in glaucoma. It is characterized by typical changes in the optic nerve head, progressive optic nerve degeneration, and loss of RGCs, if uncontrolled, leading to vision loss and blindness. The ONC injury model is often used to investigate the effects of different reagents and genes on RGC apoptosis and survival. One advantage of this model is that it has a high degree of reproducibility with minimal variations. We demonstrated here that Intravitreal delivery of trophic factors in a stable and biocompatible nanoparticle complex can protect RGCs after acute ONC in a rodent model for optic nerve injury. However, the therapeutic level of NP-trophic factors may be not achieved their optimal efficacy due to nature of the slow-releasing property in this acute optic nerve injury model. As in most cases of glaucoma, vision loss does not occur until the disease has progressed considerably and therefore glaucoma goes undiagnosed until later stages. Current treatment options are limited to lowering intraocular pressure (IOP) and can only manage the disease. Our approach provides another option for early intervention and prevention of glaucomatous diseases and optic nerve injuries.

### Example 4: P23H retinitis pigmentosa rat model experiments

Rod-cone dystrophy, also known as retinitis pigmentosa (RP), is the most common inherited degenerative photoreceptor disease, for which no therapy is currently available. The P23H opsin mutation is the most common cause of autosomal dominant RP. The P23H rat is one of the most commonly used autosomal dominant RP models. These P23H animals suffer from a progressive rod degeneration initially associated with normal cone function, and photoreceptor loss correlates with full field electroretinogram (ERG) abnormalities. These phenotypes are consistent with the clinical findings in patients carrying the P23H RHO mutation, which renders the P23H rat model a very valuable for testing therapeutic approaches for RP.

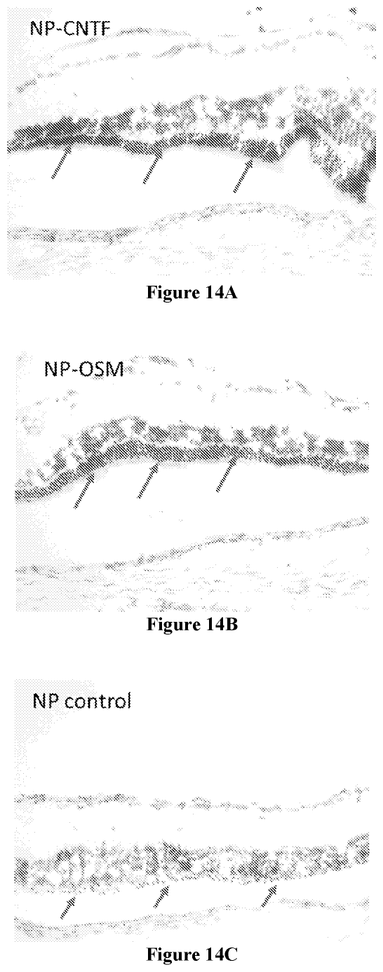

To investigate whether NP-CNTF and NP-OSM can protect photoreceptors from degeneration and preserve vision functions in the P23H rat, 4 ul of NP-CNTF, NP-OSM and control NP were intravitreal injected into one eye of P23H rats at postnatal day 20 (P20), the other eye serves as internal control. At P100, electroretinography (ERG) tests were carried out and histological analyses were performed. Significant preservation of ERG in both NP-CNTF and NP-OSM treated eyes as compared to eyes injected with control NPs (FIGS. 13A and 13B). Histological analyses showed a broad retinal neural cell preservation in all NP-CNTF and NP-OSM treated eyes (FIGS. 14A and 14B, arrow) of P23H rats, dramatically different from NP treated eyes (FIG. 14C, arrow).

### REFERENCES

Azadi, S., L. E. Johnson, F. Paquet-Durand, M. T. Perez, Y. Zhang, P. A. Ekstrom and T. van Veen (2007). “CNTF+BDNF treatment and neuroprotective pathways in the rdl mouse retina.” Brain Res 1129(1): 116-129.
Birch, D. G., L. D. Bennett, J. L. Duncan, R. G. Weleber and M. E. Pennesi (2016). “Long-term Follow-up of Patients With Retinitis Pigmentosa Receiving Intraocular Ciliary Neurotrophic Factor Implants.” Am J Ophthalmol 170: 10-14.
Buch, P. K., R. E. MacLaren, Y. Duran, K. S. Balaggan, A. MacNeil, F. C. Schlichtenbrede, A. J. Smith and R. R. Ali (2006). “In contrast to AAV-mediated Cntf expression, AAV-mediated Gdnf expression enhances gene replacement therapy in rodent models of retinal degeneration.” Mol Ther 14(5): 700-709.
Del Rio, P., M. Irmler, B. Arango-Gonzalez, J. Favor, C. Bobe, U. Bartsch, E. Vecino, J.
Beckers, S. M. Hauck and M. Ueffing (2011). “GDNF-induced osteopontin from Muller glial cells promotes photoreceptor survival in the Pde6brdl mouse model of retinal degeneration.” Glia 59(5): 821-832.
Jablonski, M. M., J. Tombran-Tink, D. A. Mrazek and A. Iannaccone (2000). “Pigment epithelium-derived factor supports normal development of photoreceptor neurons and opsin expression after retinal pigment epithelium removal.” J Neurosci 20(19): 7149-7157.
Kubin, T., J. Poling, S. Kostin, P. Gajawada, S. Hein, W. Rees, A. Wietelmann, M. Tanaka, H.
Lorchner, S. Schimanski, M. Szibor, H. Warnecke and T. Braun (2011). “Oncostatin M is a major mediator of cardiomyocyte dedifferentiation and remodeling.” Cell Stem Cell 9(5): 420-432.
Li, Y., W. Tao, L. Luo, D. Huang, K. Kauper, P. Stabila, M. M. Lavail, A. M. Laties and R.
Wen (2010). “CNTF induces regeneration of cone outer segments in a rat model of retinal degeneration.” PLoS One 5(3): e9495.
Livak, K. J. and T. D. Schmittgen (2001). “Analysis of relative gene expression data using real-time quantitative PCR and the 2(-Delta Delta C(T)) Method.” Methods 25(4): 402-408.
Lu, B., C. W. Morgans, S. Girman, R. Lund and S. Wang (2013). “Retinal morphological and functional changes in an animal model of retinitis pigmentosa.” Vis Neurosci 30(3): 77-89.
Rose-John, S. (2018). “Interleukin-6 Family Cytokines.” Cold Spring Harb Perspect Biol 10(2).
Sakai, T., N. Kuno, F. Takamatsu, E. Kimura, H. Kohno, K. Okano and K. Kitahara (2007). “Prolonged protective effect of basic fibroblast growth factor-impregnated nanoparticles in royal college of surgeons rats.” Invest Ophthalmol Vis Sci 48(7): 3381-3387.
Schwartz, S. D., J. P. Hubschman, G. Heilwell, V. Franco-Cardenas, C. K. Pan, R. M. Ostrick, E. Mickunas, R. Gay, I. Klimanskaya and R. Lanza (2012). “Embryonic stem cell trials for macular degeneration: a preliminary report.” Lancet 379(9817): 713-720.
Schwartz, S. D., C. D. Regillo, B. L. Lam, D. Eliott, P. J. Rosenfeld, N. Z. Gregori, J. P.
Hubschman, J. L. Davis, G. Heilwell, M. Spim, J. Maguire, R. Gay, J. Bateman, R. M. Ostrick, D. Morris, M. Vincent, E. Anglade, L. V. Del Priore and R. Lanza (2015). “Human embryonic stem cell-derived retinal pigment epithelium in patients with age-related macular degeneration and Stargardt's macular dystrophy: follow-up of two open-label phase 1/2 studies.” Lancet 385(9967): 509-516.
Sieving, P. A., R. C. Caruso, W. Tao, H. R. Coleman, D. J. Thompson, K. R. Fullmer and R.
A. Bush (2006). “Ciliary neurotrophic factor (CNTF) for human retinal degeneration: phase I trial of CNTF delivered by encapsulated cell intraocular implants.” Proc Natl Acad Sci USA 103(10): 3896-3901.
Sunness, J. S., J. Gonzalez-Baron, C. A. Applegate, N. M. Bressler, Y. Tian, B. Hawkins, Y. Barron and A. Bergman (1999). “Enlargement of atrophy and visual acuity loss in the geographic atrophy form of age-related macular degeneration.” Ophthalmology 106(9): 1768-1779.
Tsai, Y., B. Lu, B. Bakondi, S. Girman, A. Sahabian, D. Sareen, C. N. Svendsen and S. Wang (2015). “Human iPSC-Derived Neural Progenitors Preserve Vision in an AMD-Like Model.” Stem Cells 33(8): 2537-2549.
Walker, E. C., N. E. McGregor, I. J. Poulton, M. Solano, S. Pompolo, T. J. Fernandes, M. J.
Constable, G. C. Nicholson, J. G. Zhang, N. A. Nicola, M. T. Gillespie, T. J. Martin and N. A.
Sims (2010). “Oncostatin M promotes bone formation independently of resorption when signaling through leukemia inhibitory factor receptor in mice.” J Clin Invest 120(2): 582-592.
Wang, S., B. Lu, S. Girman, J. Duan, T. McFarland, Q. S. Zhang, M. Grompe, G. Adamus, B. Appukuttan and R. Lund (2010). “Non-invasive stem cell therapy in a rat model for retinal degeneration and vascular pathology.” PLoS One 5(2): e9200.
Xia, X., Y. Li, D. Huang, Z. Wang, L. Luo, Y. Song, L. Zhao and R. Wen (2011). “Oncostatin M protects rod and cone photoreceptors and promotes regeneration of cone outer segment in a rat model of retinal degeneration.” PLoS One 6(3): e18282.
Xia, X., R. Wen, T. H. Chou, Y. Li, Z. Wang and V. Porciatti (2014). “Protection of pattern electroretinogram and retinal ganglion cells by oncostatin M after optic nerve injury.” PLoS One 9(9): e108524.
Zaman, P., J. Wang, A. Blau, W. Wang, T. Li, D. S. Kohane, J. Loscalzo and Y. Y. Zhang (2016). “Incorporation of heparin-binding proteins into preformed dextran sulfate-chitosan nanoparticles.” Int J Nanomedicine 11: 6149-6159.
Zhang, K., J. J. Hopkins, J. S. Heier, D. G. Birch, L. S. Halperin, T. A. Albini, D. M. Brown, G. J. Jaffe, W. Tao and G. A. Williams (2011). “Ciliary neurotrophic factor delivered by encapsulated cell intraocular implants for treatment of geographic atrophy in age-related macular degeneration.” Proc Natl Acad Sci USA 108(15): 6241-6245.
Zhou, J., S. R. Kim, B. S. Westlund and J. R. Sparrow (2009). “Complement activation by bisretinoid constituents of RPE lipofuscin.” Invest Ophthalmol Vis Sci 50(3): 1392-1399.
Weinreb, R. N., Aung, T., & Medeiros, F. A. The pathophysiology and treatment of glaucoma: a review. JAMA 311, 1901-1911 (2014).
Davis, B. M., Crawley, L., Pahlitzsch, M., Javaid, F., & Cordeiro, M. F. Glaucoma: the retina and beyond. Acta Neuropathol. 132, 807-826 (2016).
Templeton, J. P and Geisert, E. E. A practical approach to optic nerve crush in the mouse.
Molecular Vision 18:2147-2152 (2012).
Tang, Z., Zhang, S., Lee, C., Kumar, A., Arjunan, P., Li, Y., Zhang, F., and Li, X. An optic nerve crush injury murine model to study retinal ganglion cell survival. Journal of Visualized Experiments 50. http://www.jove.com/details.php?id=2685, doi: 10.3791/2685 (2011)

### MODIFICATIONS

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

1. A composition for use in the treatment of a disease or condition in a subject in need thereof, comprising:
a plurality of nanoparticles comprising dextran sulfate and chitosan; and
an effective amount of a neurotrophic factor associated with the nanoparticles, wherein the neurotrophic factor is selected from the group consisting of Brain-derived neurotrophic factor (BDNF), Nerve growth factor (NGF), Neurotrophin-3 (NT-3), Neurotrophin-4 (NT-4), Ciliary neurotrophic factor (CNTF), leukemia inhibitory factor (LIF), interleukin-6 (IL-6), prolactin, growth hormone, leptin, interferons (i.e., interferon-α, -β, and -γ), oncostatin M (OSM), Glial cell line-derived neurotrophic factor (GDNF), Artemin, Neurturin, Persephin, and Ephrins.

2. The composition of claim 1, wherein the nanoparticles have an average diameter of about 200 nm to about 800 nm.

3. The composition of claim 2, wherein the nanoparticles have an average diameter of about 200 nm to about 500 nm.

4. The composition of any one of claims 1-3, wherein the dextran sulfate and chitosan are present in a weight ratio of about 10:1 to about 1:10, or about 5:1 to about 1:1.

5. The composition of claim 4, wherein the dextran sulfate and chitosan are present in a weight ratio of about 4:1.

6. The composition of any one of claims 1-3, wherein the neurotrophic factor is incorporated into the nanoparticles.

7. The composition of any one of claims 1-3, wherein the neurotrophic factor is recombinantly produced.

8. The composition of any one of claims 1-3, wherein the neurotrophic factor is CNTF and/or OSM.

9. The composition of any one of claims 1-3, wherein the neurotrophic factor is CNTF.

10. The composition of any one of claims 1-3, wherein the neurotrophic factor is OSM.

11. The composition of any one of claims 1-3, wherein the nanoparticles provide sustained and/or prolonged delivery of the neurotrophic factor.

12. The composition of claim 11, wherein the sustained and/or prolonged delivery comprises delivery for at least 6 hours, at least 12 hours, at least 24 hours, at least 48 hours.

13. The composition of any one of claims 1-3, wherein the disease or condition is a neurodegenerative disease.

14. The composition of any one of claims 1-3, wherein the disease or condition is a retinal neurodegeneration disease.

15. The composition of claim 14, wherein the retinal neurodegeneration disease is retinal degeneration and/or dystrophy (such as Leber congenital amaurosis, retinitis pigmentosa, cone-rod dystrophy, microphthalmia, anophthalmia, myopia, and hyperopia) or retinal neuronal death related diseases (such as glaucoma and age related macular degeneration, diabetic retinopathy, retinal blood vessel occlusions, retinal medication toxicity (such as what amino glycosides or plaquenil can cause), retinal trauma (e.g., post-surgical), retinal infections, choroidal dystrophies, retinal pigmentary retinopathies, inflammatory and cancer mediated auto immune diseases that result in retinal neuronal cell death).
